(19) United States
(12) Patent Application Publication
(10) Pub. No.: US 2018/0143131 A1
(43) Pub. Date: May 24, 2018

(54) PACKAGE DAMAGE INSPECTION DEVICE

(71) Applicant: KOREA FOOD RESEARCH INSTITUTE, Gyeonggi-do (KR)

(72) Inventors: Sung-Wook CHOI, Hwaseong-si, Gyeonggi-do (KR); Na-Ri LEE, Guro-gu, Seoul (KR); Hyun-Joo CHANG, Songpa-gu, Seoul (KR); Gyeong-Sik OK, Osan-si, Gyeonggi-do (KR); Ki-Sang PARK, Yongin-si, Gyeonggi-do (KR)

(21) Appl. No.: 15/579,412

(22) PCT Filed: Jun. 7, 2016
(86) PCT No.: PCT/KR2016/005994

§ 371 Date: Dec. 4, 2017

(30) Foreign Application Priority Data
Jun. 4, 2015 (KR) 10-2015-0079073
Jun. 3, 2016 (KR) 10-2016-0069795
Jun. 3, 2016 (KR) 10-2016-0069819

(51) Int. Cl. G01N 21/51; G01N 27/60; G01N 21/88; G01M 3/38; G01N 27/61; G01N 23/20; G01N 21/3586

(52) U.S. Cl. CPC G01N 21/51; G01N 27/605; G01N 21/8806; G01N 33/02; G01N 27/61; G01N 23/20; G01N 21/3586; G01M 3/38

(57) ABSTRACT

A package damage inspection device according to an exemplary embodiment of the present invention may include: a sensor recognizing internal environmental change information of a sealed container; an identification element including an identification code of the sealed container; a recognizing unit recognizing the identification code included in the identification element to recognize identification information of the sealed container; and a determining unit comparing the internal environmental change information recognized by the sensor and reference change information with each other to determine whether or not the sealed container is maintained in a sealed state.

### TECHNICAL FIELD

The present invention relates to a device capable of conveniently inspecting whether or not a package is damaged using a sensor.

The present invention relates to a humidity sensor using a guided mode resonance element, and a container inspection device using the same.

### BACKGROUND ART

Recently, a problem that a food, or the like, included in a package is changed or crushed due to damage to the package in a process of manufacturing the food, a process of distributing the food, or the like, has frequently occurred. In the case of the food, the package has been damaged in various situations such as a situation in which a worm penetrates through and enters the package in the process of manufacturing the food, the process of distributing the food, or the like, a situation in which the package drops in a delivery process, a situation in which a person touches a displayed product, and a situation in which a person maliciously injects a poison into the package.

As a method of inspecting the damage to the package generated in the process of manufacturing the food or the process of distributing the food, \textcircled{1} a method of inspecting the damage to the package with the naked eyes in the process of manufacturing the food, \textcircled{2} a method of inspecting whether or not air bubbles are generated in the process of manufacturing the food, and \textcircled{3} a vision inspection method using a camera, and \textcircled{4} an inspection method using a pin hole detector using various manners have been used.

Technology related to the related art is disclosed in Korean Patent Laid-Open Publication No. 10-2008-0014240 (entitled “Pin-hole Detecting Device and Method of Vessel”).

Generally, various products that are distributed and sold have been sold in a package state, such that it is possible to confirm a direct damage state of a container with the naked eyes, but it is difficult to confirm state information in the container. Particularly, in the case of products in which serious damage is generated when moisture exists in the container, such as food products or electronic products, it is further required to sense the moisture existing in the container.

There may be a method of recognizing humidity information by disposing an electrically driven humidity sensor in the container in order to solve this.

Since a general humidity sensor has a production cost higher than that of the container, it may be used in electronic products that are expensive, but it is difficult in terms of productivity to use the general humidity sensor in general food products.

Therefore, there is a need to develop a humidity sensor that may be used in products in various package states due to a low cost.

### DISCLOSURE

### Technical Problem

An object of the present invention is to provide a package damage inspection device capable of easily and accurately inspecting whether or not a package is damaged using a sensor and accurately figuring out in which process damage to the package is generated by inspecting whether or not the package is damaged in all distribution processes.

Other objects and advantages of the present invention may be understood by the following description and will be more clearly appreciated by exemplary embodiments of the present invention. In addition, it may be easily appreciated that objects and advantages of the present invention may be realized by means mentioned in the claims and a combination thereof.

### Technical Solution

According to an exemplary embodiment of the present invention, a package damage inspection device may include: a sensor recognizing internal environmental change information of a sealed container; an identification element including an identification code of the sealed container; a recognizing unit recognizing the identification code included in the identification element to recognize identification information of the sealed container; and a determining unit comparing the internal environmental change information recognized by the sensor and reference change information with each other to determine whether or not the sealed container is maintained in a sealed state.

The determining unit may compare the internal environmental change information recognized by the sensor and the reference change information with each other to determine whether or not a change in an internal environment of the sealed container exists, and determine that the sealed container is not maintained in the sealed state in the case in which it is determined that the change in the internal environment exists.

The determining unit may compare internal environmental change information generated in a current measuring step and internal environmental change information generated in the previous measuring step with each other to determine that the sealed container is not maintained in the sealed state.

The determining unit may compare internal environmental change information generated in a current measuring step and current external environment information with each other to determine whether or not the sealed container is maintained in the sealed state.

The internal environmental change information may be at least one of temperature information, humidity information, information on whether or not a specific material included in sealing exists, and concentration information of a material included in the sealing.

The sensor may be provided in the container, and may periodically transmit the internal environmental change information to the determining unit through a communication unit or transmit the internal environmental change information to the determining unit through the communication unit whenever a request signal is input.

The package damage inspection device may further include an information generating unit generating at least one of internal environmental change information for each identification code and each distributing step, decision result information on whether or not an internal environmental change exists, external environment information at the time of performing measurement, information on a measurement day and time, and information on a measurer.

The information generating unit may transmit the generated information and a warning message to an external device through a communication unit.

The identification element may be any one of a bar code, a quick response (QR) code, and a radio frequency identification (RFID) code, and the recognizing unit may be a device recognizing any one of the bar code, the QR code, and the RFID code.

The package damage inspection device may further include an electromagnetic wave generating unit generating an electromagnetic wave, wherein the sensor is provided in the sealed container, and changes an electromagnetic wave incident thereto depending on an internal environmental change of the sealed container to generate the changed electromagnetic wave, and the determining unit compares the electromagnetic wave generated from the sensor and a reference electromagnetic wave corresponding to the identification code with each other to determine whether or not the sealed container is maintained in the sealed state.

The identification element may be an optical identification element generating a natural resonant frequency when the electromagnetic wave is incident thereto.

The package damage inspection device may further include a detecting unit detecting characteristics of the electromagnetic wave generated from the sensor and detecting the natural resonant frequency of the electromagnetic wave generated from the optical identification element, wherein the recognizing unit recognizes the identification code of the container on the basis of the detected natural resonant frequency, and the determining unit compares the electromagnetic wave generated from the container and a reference electromagnetic wave corresponding to the identification code with each other to determine whether or not the sealed container is maintained in the sealed state.

The determining unit may determine the sealed container is not maintained in the sealed state in the case in which a difference value between the natural resonant frequency of the electromagnetic wave generated from the container and a natural resonant frequency of the reference electromagnetic wave is greater than a set difference value.

The reference change information may have reference change information different from each other in each of a first distributing step, a second distributing step, and an N-th distributing step, and the determining unit may compare the internal environmental change information recognized by the sensor and reference change information corresponding to the identification code of the sealed container and corresponding to a current distributing step with each other to determine whether or not the sealed container is maintained in the sealed state.

The optical identification element may include m identification units, and each of the identification units may include an electromagnetic wave transmitting layer formed of a material transmitting the electromagnetic wave therethrough and a waveguide diffraction grating generating resonance in a natural resonant frequency when the transmitted electromagnetic wave is irradiated thereto, the natural resonant frequency being any one of a first natural resonant frequency to an n-th natural resonant frequency.

Since a kind of unique resonance frequencies is n and the number of identification units is m, the number of identification codes that is represented by the optical identification element may be n^{m}.

The package damage inspection device may further include a writing unit writing at least one of the internal environmental change information for each identification code and each distributing step, decision result information on whether or not the sealed container is maintained in the sealed state, the external environment information at the time of performing the measurement, the information on the measurement day and time, and the information on the measurer in the identification element, wherein the recognizing unit recognizes the information included in the identification element.

According to another exemplary embodiment of the present invention, a package damage inspection device may include: a sensor recognizing internal environmental change information of a sealed container; an identification element for a terahertz wave including m identification units including a terahertz wave transmitting layer formed of a material transmitting a terahertz wave therethrough, a waveguide diffraction grating generating resonance in a natural resonant frequency when the transmitted terahertz wave is irradiated thereto, and an identification code of the sealed container, the natural resonant frequency being any one of a first natural resonant frequency to an n-th natural resonant frequency; a recognizing unit recognizing the identification code included in the identification element for a terahertz wave to recognize identification information of the sealed container; and a determining unit comparing the internal environmental change information recognized by the sensor and reference change information with each other to determine whether or not the sealed container is maintained in a sealed state.

The package damage inspection device may further include a light source irradiating the terahertz wave to the identification element for a terahertz wave, wherein the recognizing unit detects unique resonance frequencies of the respective terahertz waves generated from the respective identification elements for a terahertz wave, and recognizes the identification code on the basis of the detected unique resonance frequencies.

The sensor may include a terahertz wave transmitting layer formed of a material transmitting the terahertz wave therethrough and an electric field enhancing structure reacting to a preset frequency band in the terahertz wave transmitted through the terahertz wave transmitting layer to enhance an electric field.

The determining unit may compare the internal environmental change information recognized by the sensor and the reference change information with each other to determine whether or not a change in an internal environment of the sealed container exists, and determine that the sealed container is not maintained in the sealed state in the case in which it is determined that the change in the internal environment exists.

The determining unit may compare internal environmental change information generated in a current measuring step and internal environmental change information generated in the previous measuring step with each other to determine that the sealed container is not maintained in the sealed state.

The determining unit may compare internal environmental change information generated in a current measuring step and current external environment information with each other to determine whether or not the sealed container is maintained in the sealed state.

The package damage inspection device may further include a writing unit for an identification unit writing at least one of decision result information on whether or not the sealed container is maintained in the sealed state, external environment information at the time of performing measurement, information on a measurement day and time, and a information on a measurer in the identification unit.

According to still another exemplary embodiment of the present invention, a package damage inspection system may include: a package damage inspection device including a sensor recognizing internal environmental change information of a sealed container, an identification element including an identification code of the sealed container, a recognizing unit recognizing the identification code included in the identification element to recognize identification information of the sealed container, and a determining unit comparing the internal environmental change information recognized by the sensor and reference change information with each other to determine whether or not the sealed container is maintained in a sealed state; and a server receiving information on whether or not the sealed container is maintained in the sealed state from the package damage inspection device and storing the received information on whether or not the sealed container is maintained in the sealed state in a storing unit or transmitting the received information on whether or not the sealed container is maintained in the sealed state to a terminal of a manager.

The package damage inspection device may be provided in each of a first distributing step, a second distributing step, and an N-th distributing step, and the server may receive the information on whether or not the sealed container is maintained in the sealed state, internal environmental change information for each identification code and each distributing step, external environment information at the time of performing measurement, information on a measurement day and time, and information on a measurer from the package damage inspection device in each of the first distributing step, the second distributing step, and the N-th distributing step.

The determining unit may compare the internal environmental change information recognized by the sensor and reference change information corresponding to the identification code of the sealed container and corresponding to a current distributing step with each other to determine whether or not the sealed container is maintained in the sealed state.

The determining unit may compare the internal environmental change information recognized by the sensor and the reference change information with each other to determine whether or not a change in an internal environment of the sealed container exists, and determine that the sealed container is not maintained in the sealed state in the case in which it is determined that the change in the internal environment exists.

The determining unit may compare internal environmental change information generated in a current measuring step and internal environmental change information generated in the previous measuring step with each other to determine that the sealed container is not maintained in the sealed state.

The determining unit may compare internal environmental change information generated in a current measuring step and current external environment information with each other to determine whether or not the sealed container is maintained in the sealed state.

According to yet still another exemplary embodiment of the present invention, a humidity sensor using a guided mode resonance element may include: a guide mode resonance (GMR) element; and a moisture sensing film applied to the GMR element so as to absorb moisture and formed to change a second electromagnetic wave generated in the GMR element depending on an attenuation coefficient by the moisture in the case in which a first electromagnetic wave is irradiated from the outside to the GMR element.

The moisture sensing film may be formed to change a reflectance of the second electromagnetic wave.

The moisture sensing film may be formed to change a quality factor (Q=f/Δf (here, f is a resonance frequency and Δf is a full width half maximum frequency)) of the second electromagnetic wave.

The moisture sensing film may be formed of an inorganic material including at least any one of lithium chloride, silica gel, and activated alumina.

The moisture sensing film may be formed of an organic material including at least any one of a carboxyl group (—COOH), an amine group (—NH2), and an alcohol group (—OH).

The GMR element may include a grating layer formed in one direction, and the moisture sensing film may be applied onto the grating layer.

According to yet still another exemplary embodiment of the present invention, a container inspection device may include: a humidity sensor using a GMR element, including: the GMR element and a moisture sensing film applied to the GMR element so as to absorb moisture and formed to change a second electromagnetic wave generated in the GMR element depending on an attenuation coefficient by the moisture in the case in which a first electromagnetic wave is irradiated from the outside to the GMR element; a light source irradiating the first electromagnetic wave to the humidity sensor; a detecting unit detecting the second electromagnetic wave generated from the humidity sensor; and a humidity information generating unit generating humidity information on the basis of the second electromagnetic wave detected from the detecting unit.

The humidity information generating unit may generate the humidity information on the basis of at least any one of a reflectance and a quality factor of the second electromagnetic wave.

The container inspection device may further include a user input unit formed to input component information, thickness information, and reflective index information of the moisture sensing film, and frequency information of the first electromagnetic wave.

The humidity information generating unit may generate the humidity information on the basis of at least one of the thickness information, the component information, the reflective index information, and the frequency information.

The container inspection device may further include a display unit formed to output the humidity information.

The moisture sensing film may be formed of an inorganic material including at least any one of lithium chloride, silica gel, and activated alumina.

The moisture sensing film may be formed of an organic material including at least any one of a carboxyl group (—COOH), an amine group (—NH2), and an alcohol group (—OH).

The GMR element may include a grating layer formed in one direction, and the moisture sensing film may be applied onto the grating layer.

### Advantageous Effects

According to the present invention, it is determined whether or not the sealed container is maintained in the sealed state using the sensor provided in the sealed container to inspect whether or not the container is damaged, thereby making it possible to inspect whether or not the container is damaged by a non-destructive method.

In addition, it is inspected whether or not the package is damaged in all distribution processes, thereby making it possible to accurately figure out in which process the damage to the package is generated.

Further, the information on whether or not the package is damaged is transmitted to a user, a manager, and a managing server, thereby making it possible to inform the user, the manager, and the managing server of the information on whether or not the package is damaged in real time and allow the information on whether or not the package is damaged to be collectively managed by the managing server.

Further, it is inspected whether or not the package is damaged on the basis of changed characteristics of the electromagnetic wave, thereby making it possible to accurately inspect whether or not the package is damaged.

The humidity sensor using a GMR element and the container inspection device using the same according to the present invention may be used in various products due to a low cost.

In addition, it may be determined whether or not moisture is sensed on the basis of a change in the quality factor depending on the attenuation coefficient by the moisture.

Further, convenience in inspecting the container may be further improved through a simple operation of recognizing the humidity sensor as an inspection device.

### DESCRIPTION OF DRAWINGS

FIG. 1 is a view for describing a package damage inspection device for a sealed container according to an exemplary embodiment of the present invention.

FIG. 2 is a view for describing a package damage inspection device according to another exemplary embodiment of the present invention.

FIGS. 3A to 3E are views for describing applications of the package damage inspection device according to an exemplary embodiment of the present invention.

FIGS. 4A to 4C are views for describing a driving example of the package damage inspection device according to an exemplary embodiment of the present invention.

FIGS. 5A to 5C are views for describing a driving example of a package damage inspection device according to another exemplary embodiment of the present invention.

FIGS. 6A to 6C are views for describing a driving example of a package damage inspection device according to still another exemplary embodiment of the present invention.

FIG. 7 is a view for describing a container according to an exemplary embodiment of the present invention.

FIG. 8 is a view for describing a container according to another exemplary embodiment of the present invention.

FIG. 9 is a view for describing a sensor included in the container according to an exemplary embodiment of the present invention.

FIGS. 10A to 10C are views for describing an electric field enhancing structure according to an exemplary embodiment of the present invention.

FIG. 11 is a view for describing an optical identification element according to an exemplary embodiment of the present invention.

FIGS. 12A to 12D are views for describing the optical identification element according to an exemplary embodiment of the present invention in detail.

FIGS. 13A to 13C are views for describing a writing device for an identification unit according to an exemplary embodiment of the present invention.

FIG. 14 is a view for describing a package damage inspection system according to an exemplary embodiment of the present invention.

FIG. 15 is a side view for describing a structure of a humidify sensor 400 according to an exemplary embodiment of the present invention.

FIGS. 16A and 16B are views for describing an operation method of the humidify sensor 400 of FIG. 1.

FIG. 17 is a view for describing a change in a quality factor of a second electromagnetic wave depending on a change in an attenuation coefficient of a moisture sensing film 430.

FIG. 18 is a view for describing a change in a reflectance of a second electromagnetic wave depending on a change in an attenuation coefficient of the moisture sensing film 430.

FIG. 19 is a view for describing a container inspection device 200 according to another exemplary embodiment of the present invention.

### BEST MODE

Hereinafter, detailed contents for embodying the present invention will be described in detail with reference to the accompanying drawings.

FIG. 1 is a view for describing a package damage inspection device for a sealed container according to an exemplary embodiment of the present invention.

Referring to FIG. 1, the package damage inspection device 10 for a sealed container includes a display unit 11, a storing unit 12, a communication unit 13, a recognizing unit 14, a determining unit 15, an information generating unit 16, and a writing unit 17.

The sealed container means a container to which a packaging method for blocking from an external environment after packaging such as vacuum packaging, gas filling packaging, or the like, is applied.

The display unit 11 may display various data information. As an example, the display unit 11 may display information on whether or not an internal environmental change of a container exists.

The display unit 11 may include at least one of a liquid crystal display (LCD), a thin film transistor-liquid crystal display (TFT LCD), an organic light-emitting diode (OLED), a flexible display, and a 3D display.

The storing unit 12 may store the various data information. As an example, the storing unit 12 may store identification code information, internal environmental change information, decision result information on whether or not the internal environmental change exists, and the like.

The communication unit 13 may transmit and receive various information. As an example, the communication unit 13 may receive the internal environmental change information or the identification code information transmitted from a sensor 21 and an identification element 22. As another example, the communication unit 13 may transmit various information to an external terminal or a managing server.

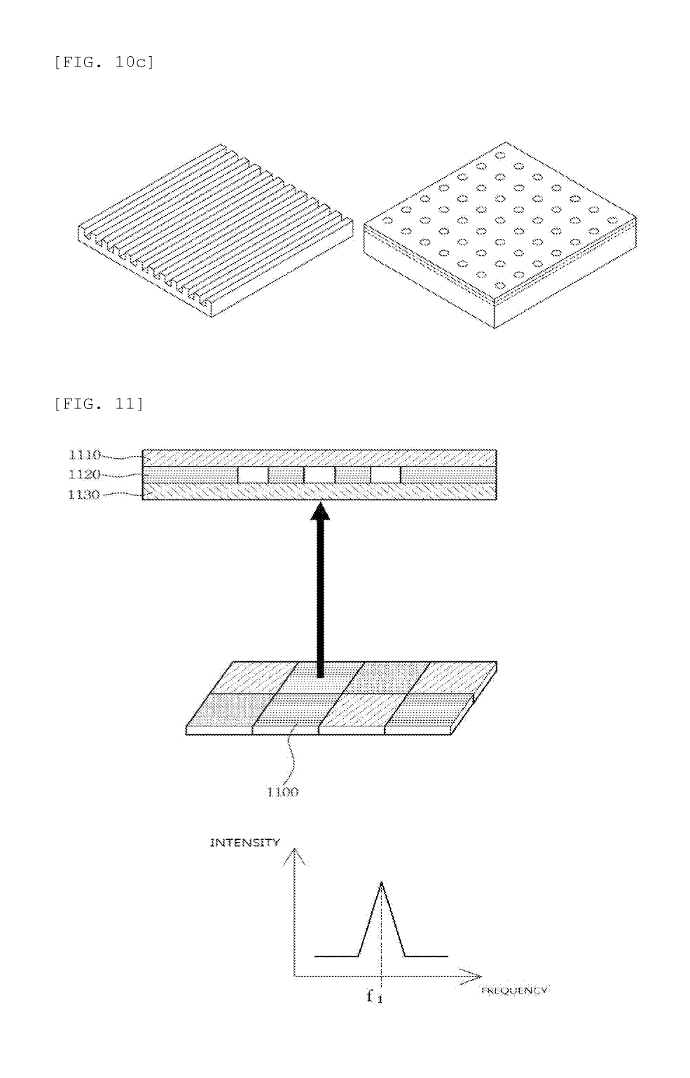

The recognizing unit 14 may recognize an identification code included in the identification element 22. The identification codes may be differently given to each container. As an example, the identification element 22 may be any one of a bar code, a quick response (QR) code, and a radio frequency identification (RFID) code, and the recognizing unit 14 may be a device that may recognize any one of the bar code, the QR code, and the RFID code. As another example, the identification element may be an optical identification element. The optical identification element will be described in detail with reference to FIG. 11.

The determining unit 15 may compare the internal environmental change information recognized by the sensor 21 and reference change information with each other to determine whether or not the sealed container is maintained in a sealed state. As an example, the determining unit 15 may compare the internal environmental change information recognized by the sensor 21 and the reference change information with each other to determine whether or not a change in an internal environment of the sealed container exists. The determining unit 15 may determine that the sealed container is not maintained in the sealed state in the case in which it is determined that the change in the internal environment exists. That is, the determining unit 15 may determine that a sealed portion of the sealed container is damaged.

The determining unit 15 may know the change in the internal environment of the container including physical, chemical, and biological changes. The internal environment change information may include physical change information, chemical change information, biological change information, and the like. The physical change may mean a change in a temperature, a volume, a form, or the like, the chemical change may mean a quantitative change for a component such as a material, a gas, moisture, and the like, and the biological change may mean a change in the number of population, such as microorganisms, viruses, fungi, or the like. A level of the change may be determined by a difference level between the internal environmental change information recognized by the sensor 21 and reference change information. The reference change information may be a reference value for determining the internal environmental change information set for each identification code and each distributing step.

The determining unit 15 may compare internal environmental change information generated in a current distributing step and internal environmental change information generated in the previous distributing step with each other to determine whether or not the sealed container is maintained in the sealed state. As an example, in the case in which a humidity in the previous distributing step is A, when a humidity generated in the current distributing step is A, the determining unit 15 may determine that the internal environmental change does not exist. On the other hand, when a humidity generated in the current distributing step is B, the determining unit 15 may determine that the internal environmental change exists to determine that a sealed package portion is damaged. In other words, the determining unit 15 may compare internal environmental change information in a current sealed state and internal environmental change information in a normal sealed state with each other to determine whether or not the sealed package portion is damaged (‘whether or not the sealed container is maintained in the sealed state’). The present invention uses a phenomenon in which the internal environment of the sealed container is changed from an internal environment of the container at the time of being initially sealed in the case in which the sealed package portion is damaged.

The information generating unit 16 may generate information on the internal environmental change of the container for each identification code and each distributing step, external environment information at the time of performing measurement, information on a measurement day and time, information on a measurer, and the like.

The information generating unit 16 may transmit the generated information and a warning message to an external device such as a terminal used by a user managing damage to the package in distribution, a managing server, or the like, through the communication unit 13 or store the generated information in the storing unit 12. Therefore, a manager may confirm whether or not the package is damaged in the distribution in real time.

The writing unit 17 may write various information measured in a current step in the identification element 22. Here, the various information may include the internal environmental change information set for each identification code and each distributing step, decision result information on whether or not the sealed container is maintained in the sealed state, the external environment information at the time of performing the measurement, the information on the measurement day and time, the information on the measurer, and the like.

As described above, the wiring unit 17 writes the various information in the identification element 22, such that the recognizing unit 14 may recognize the various information measured in the previous step, included in the identification element 22. Therefore, the recognizing unit 14 does not receive the various information measured in the previous step from a server (not illustrated), or the like, but may directly recognize the various information measured in the previous step from the identification element 22.

The sensor 21 may be attached to the sealed container 20, be provided integrally with the sealed container 20, or be provided in the sealed container 20.

As an example, in the case in which a wave is incident from the outside to the sensor 21, the sensor 21 may change the incident wave depending on the internal environmental change to generate the changed wave. Therefore, the sensor 21 may recognize the internal environmental change information by considering a feature of the changed wave. Here, the wave may include an electromagnetic wave, an ultrasonic wave, and the like.

As another example, the sensor 21 may periodically transmit the internal environmental change information to the determining unit 15 through a communication unit (not illustrated) or transmit the internal environmental change information to the determining unit 15 through the communication unit (not illustrated) whenever a request signal is input. As an example, the sensor 21 may be a passive type sensor that does not include a separate power supply or an active type sensor that includes a separate power supply and may actively transmit the internal environmental change information to the determining unit 15.

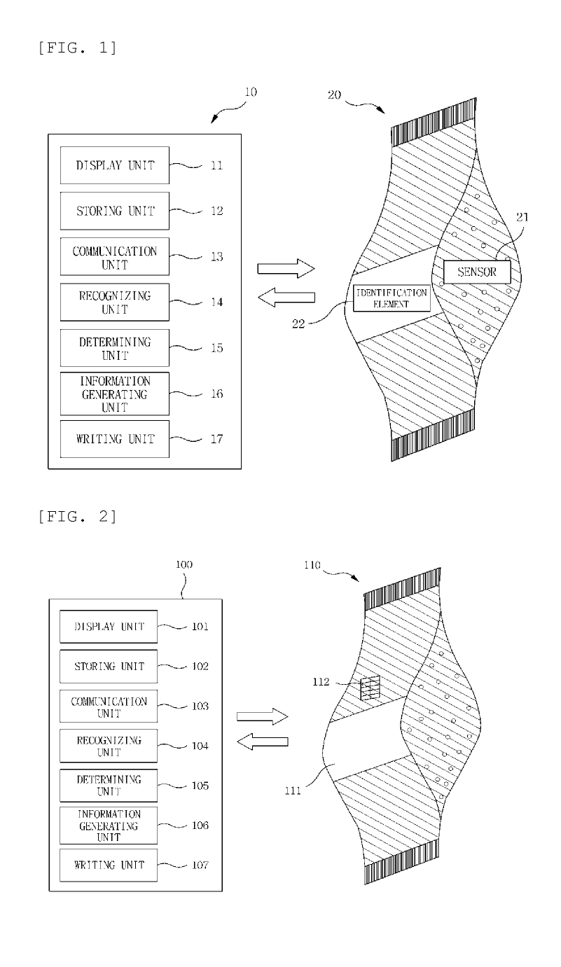

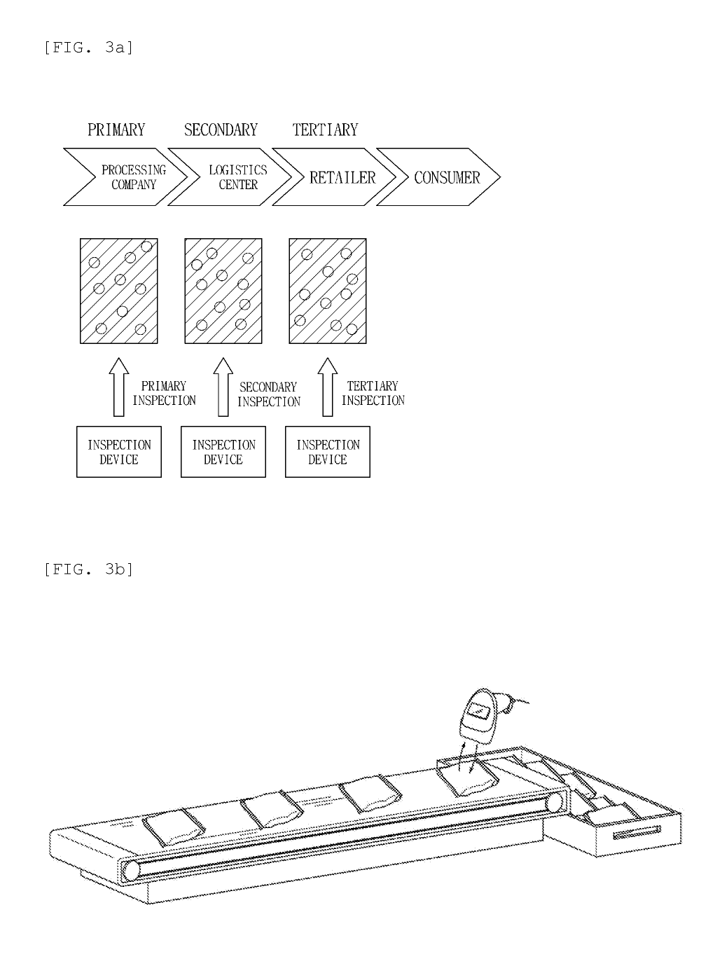

FIG. 2 is a view for describing a package damage inspection device according to another exemplary embodiment of the present invention.

Referring to FIG. 2, the package damage inspection device 100 includes a display unit 101, an electromagnetic wave generating unit 102, a detecting unit 103, a recognizing unit 104, a determining unit 105, an information generating unit 106, and a writing unit 107.

The display unit 101 may display various data information. As an example, the display unit 102 may display information on whether or not an internal environmental change of a container exists.

The display unit 101 may include at least one of a liquid crystal display (LCD), a thin film transistor-liquid crystal display (TFT LCD), an organic light-emitting diode (OLED), a flexible display, and a 3D display.

The electromagnetic wave generating unit 102 may irradiate an electromagnetic wave to a container 111 and an optical identification element 112. As an example, the electromagnetic wave generating unit 102 may be various types of devices that may generate a terahertz wave. The terahertz wave, which is an electromagnetic wave positioned in a region between an infrared ray and a microwave, may generally have a frequency of 0.1 THz to 10 THz. However, even though the terahertz wave is slightly out of the range described above, the terahertz wave may be considered as the terahertz wave in the present invention when it is in a range that may be easily deduced by those skilled in the art to which the present invention pertains. The container 111 may change the electromagnetic wave incident from an external electromagnetic wave generating unit depending on an internal environmental change thereof to generate the changed electromagnetic wave. The optical identification element 112 may generate a natural resonant frequency when the electromagnetic wave is incident thereto. A detailed description for the container 111 and the optical identification element 112 will be provided below.

The detecting unit 103 may detect characteristics of the terahertz wave generated from the container 111, and detect the natural resonant frequency of the electromagnetic wave generated from the optical identification element 112.

The detecting unit 103 may detect characteristics of the terahertz wave reflected from, transmitted through, diffracted from, or scattered from the container 111. In a specific example, the detecting unit 103 may detect intensity, a resonance frequency, or the like, of the terahertz wave generated from the container 111 for terahertz.

The detecting unit 103 may detect a natural resonant frequency of the electromagnetic wave generated from the optical identification element 112.

The recognizing unit 104 may recognize an identification code of the container 111 on the basis of the natural resonant frequency detected by the detecting unit 103. The identification code may include code information that may distinguish a plurality of containers from each other.

The determining unit 105 may compare the electromagnetic wave generated from the container 111 detected by the detecting unit 103 and a reference electromagnetic wave corresponding to the identification code with each other to determine whether or not a sealed container is maintained in a sealed state. As an example, the determining unit 105 may know an internal environmental change of the container including physical, chemical, and biological changes. The physical change may mean a change in a temperature, a volume, a form, or the like, the chemical change may mean a quantitative change for a component such as a material, a gas, moisture, and the like, and the biological change may mean a change in the number of population, such as microorganisms, viruses, fungi, or the like. A level of the change may be determined depending in a difference level between a resonance frequency of the electromagnetic wave detected from the container 111 and a resonance frequency of a reference terahertz wave.

The reference electromagnetic waves for each identification code and each distributing step may have different values.

As an example, the determining unit 105 may determine that the internal environmental change of the container 111 exists in the case in which a difference value between the resonance frequency of the electromagnetic wave detected by the detecting unit 103 and the resonance frequency of the reference electromagnetic wave corresponding to the recognized identification code is greater than a set difference value.

TABLE 1

| | Reference | Reference | Reference |
|---|---|---|---|
| | Resonance | Resonance | Resonance |
| | Frequency in | Frequency in | Frequency in |
| | Processing | Delivering | Purchasing |
| Division | Step | Step | Step |
| Identification Code 1 | A | B | C |
| Identification Code 2 | D | E | F |
| Identification Code 3 | G | H | I |
| Identification Code 4 | J | K | L |

Referring to Table 1, reference resonance frequencies for each step of Identification Code 1 may be A, B, and C. The reference resonance frequencies may be preset or be resonance frequency values of electromagnetic waves measured in the previously step. As an example, in the case in which a resonance frequency of an electromagnetic wave actually detected from the container 111 in the processing step is B, a reference resonance frequency in the delivering step may be set to B. In addition, in the case in which a resonance frequency of an electromagnetic wave actually detected from the container 111 in the delivering step is C, a reference resonance frequency in the purchasing step may be set to C. In this case, the determining unit 105 may determine whether or not the internal environmental change of the container 111 exists on the basis of B−A (a difference value) in the processing step, determine whether or not the internal environmental change of the container 111 exists on the basis of C−B (a difference value) in the delivering step, and determine whether or not the sealed container 111 is maintained in the sealed state on the basis of D−C (a difference value) in the purchasing step.

As another example, the determining unit 105 may determine whether or not the sealed container 111 is maintained in the sealed state on the basis of a difference value between the resonance frequency of the electromagnetic wave detected by the detecting unit 103 and the resonance frequency of the reference terahertz wave corresponding to the recognized identification code. As a specific example, the determining unit 105 may digitize levels of internal physical, chemical, and biological changes of the container 111 on the basis of the difference value. As an example of a humidity, the determining unit 105 may derive a difference value of a humidity on the basis of the difference value.

As another example, the determining unit 105 may compare intensity of the terahertz wave detected by the detecting unit 103 in a specific wavelength and intensity of the reference terahertz wave corresponding to the recognized identification code with each other, and determine that the sealed container 111 is damaged from the sealed state in the case in which a difference value between the intensity of the terahertz wave detected by the detecting unit 103 in the specific wavelength and the intensity of the reference terahertz wave corresponding to the recognized identification code is greater than a set difference value.

The information generating unit 106 may generate information on the internal environmental change of the container for each identification code and each distributing step, external environment information at the time of performing measurement, information on a measurement day and time, information on a measurer, and the like.

The information generating unit 16 may transmit the generated information and a warning message to an external device such as a terminal used by a user managing damage to the package in distribution, a managing server, or the like, through the communication unit. Therefore, a manager may confirm whether or not the package is damaged in the distribution in real time.

The writing unit 107 may write various information measured in a current step and including decision result information on whether or not the sealed container is maintained in the sealed state, the environment information at the time of performing the measurement, the external information on the measurement day and time, the information on the measurer, and the like, in the identification element 112.

The package damage inspection device may determine whether or not the sealed container is maintained in the sealed state using the sensor provided in the sealed container to inspect whether or not the container is damaged, thereby making it possible to inspect whether or not the container is damaged using a non-destructive method.

In addition, the package damage inspection device may inspect whether or not the container is damaged on the basis of changed characteristics of the electromagnetic wave to accurately inspect whether or not the container is damaged.

Further, the package damage inspection device may inspect whether or not the package is damaged in all distribution processes to accurately inspect in which process the package is damaged.

Further, the package damage inspection device may transmit information on whether or not the package is damaged to the user, the manager, and the managing server, to inform the user, the manager, and the managing server of the information on whether or not the package is damaged in real time and allow the information on whether or not the package is damaged to be collectively managed by the managing server.

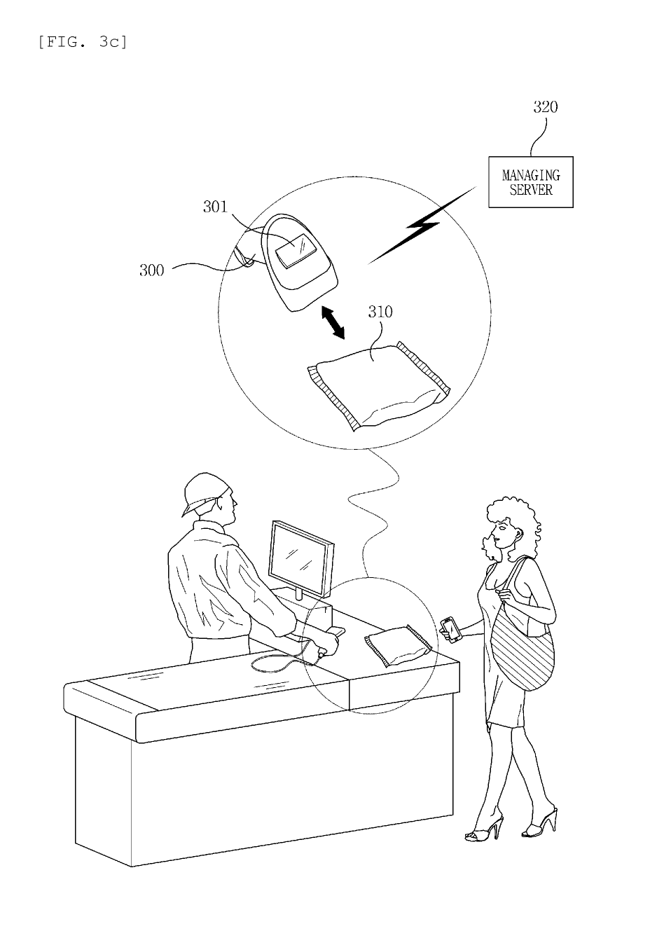

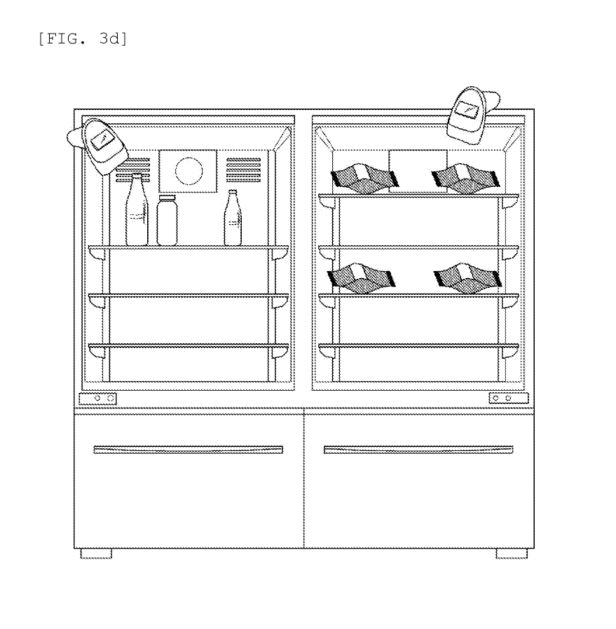

FIGS. 3A to 3E are views for describing applications of the package damage inspection device according to an exemplary embodiment of the present invention.

Referring to FIGS. 1 and 3A, the user, or the like, may inspect whether or not the sealed container is maintained in the sealed state per distributing step using the package damage inspection device 10.

The package damage inspection device 10 may obtain information on whether or not the sealed container is maintained in the sealed state, external environment information, information on a measurement day and time, information on a measurer, and the like, for each identification code and each distributing step.

Referring to FIGS. 1 and 3B, the package damage inspection device 10 may obtain information on whether or not the sealed container is maintained in the sealed state, external environment information, information on a measurement day and time, information on a measurer, and the like, for each identification code and each distributing step, for each sealed container in a primary step, which is a producing step of a processing company.

Referring to FIGS. 1 and 3C, the package damage inspection device 10 may obtain information on whether or not the sealed container is maintained in the sealed state, external environment information, information on a measurement day and time, information on a measurer, and the like, for each identification code and each distributing step, for each sealed container in a secondary step, which is a distributing step of a logistics center, or a tertiary step, which a selling step.

Referring to FIGS. 1 and 3D, the package damage inspection device 10 may obtain information on whether or not the sealed container is maintained in the sealed state (‘information on whether or not the change exists’), external environment information, information on a measurement day and time, information on a measurer, and the like, for each identification code and each distributing step, for each sealed container in a quaternary step, which is a storing step of a consumer. For example, the package damage inspection device 10 may be provided in a refrigerant, and may determine whether or not the sealed container is maintained in the sealed state through an inspection for each sealed container.

Referring to FIGS. 1 and 3E, the package damage inspection device 10 may store the obtained information on whether or not the sealed container is maintained in the sealed state (‘information on whether or not the change exists’), external environment information, information on a measurement day and time, information on a measurer, and the like, for each identification code and each distributing step in the storing unit 12, display them on the display unit 11, or transmit them in real time to an external device through the communication unit 13.

Therefore, states of the sealed containers for each identification code and each distributing step may be collectively confirmed.

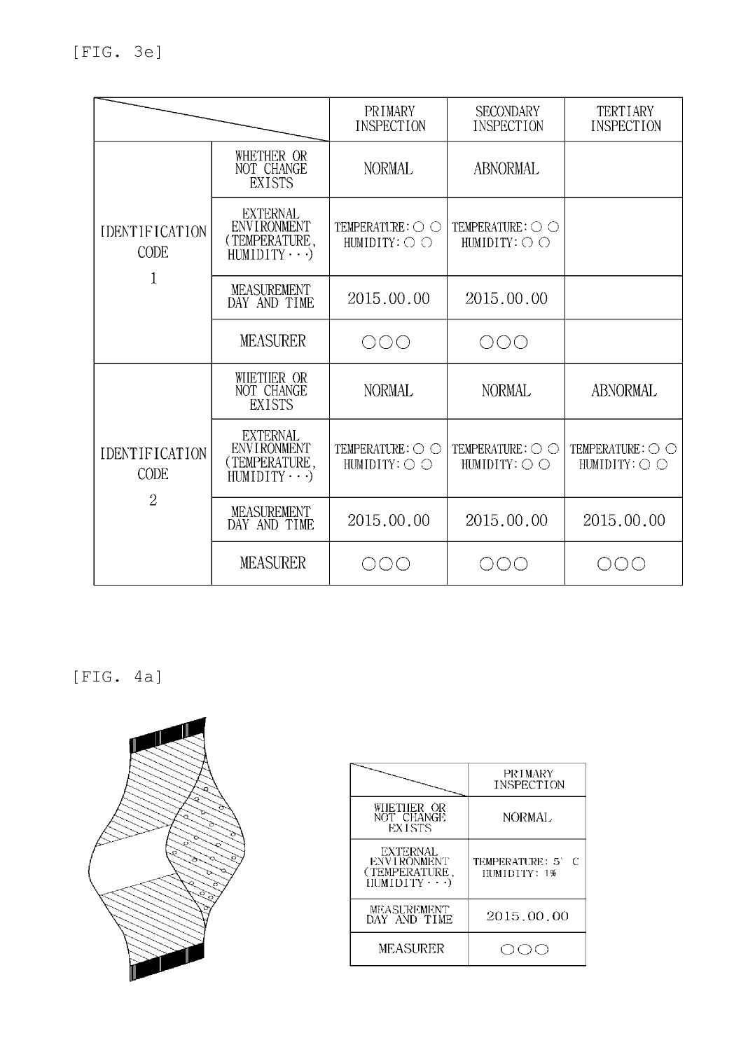

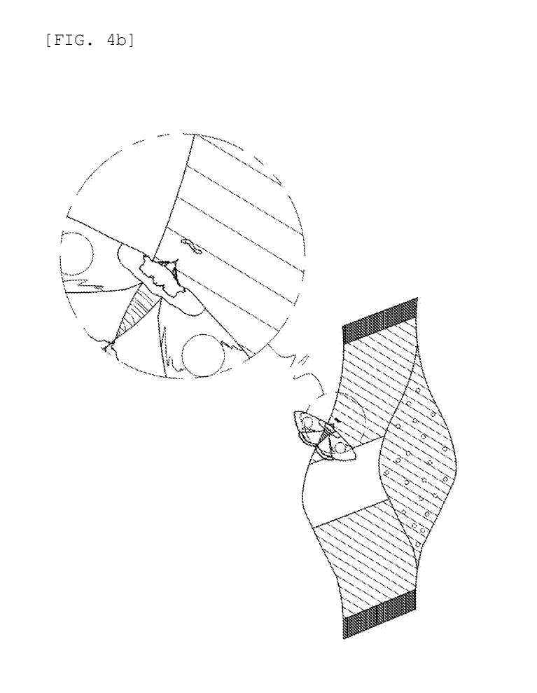

FIGS. 4A to 4C are views for describing a driving example of the package damage inspection device according to an exemplary embodiment of the present invention.

Referring to FIG. 4A, the package damage inspection device may obtain information in a manufacturing step. For example, the information may include information on whether or not the sealed container is maintained in the sealed state (‘information on whether or not the change exists’), internal environmental change information (a humidity, a temperature, a specific gas concentration, and the like), information on a measurement day and time, and information on a measurer. In the case in which the sealed container is in a normal state, a temperature may be 5° C. and a humidity may be 1%.

Referring to FIG. 4B, in a distributing step, a selling step, or the like, a moth may damage the sealed container and lay eggs in the sealed container. A portion damaged by the moth is very fine, such that it may not be determined whether or not the sealed container is damaged with the naked eyes.

Referring to FIG. 4C, the user, or the like, may inspect the sealed container using the package damage inspection device after the manufacturing step. In the case in which a temperature is 20° C. and a humidity is 7% as an inspection result, the package damage inspection device may determine that the sealed container is damaged from the sealed state since a difference of internal environmental change information is large as compared with the normal state (‘temperature: 5° C. and humidity: 1%’).

When sealing is finely damaged as in the present exemplary embodiment, entry of an external gas, liquid, or solid is allowed, such that an internal environment of the sealed container tends to become similar to an external environment.

Even in the case in which the sealing is finely damaged by the moth as described above, when the package damage inspection device according to the present invention is used, it may be easily determined whether or not the sealed container is maintained in the sealed state.

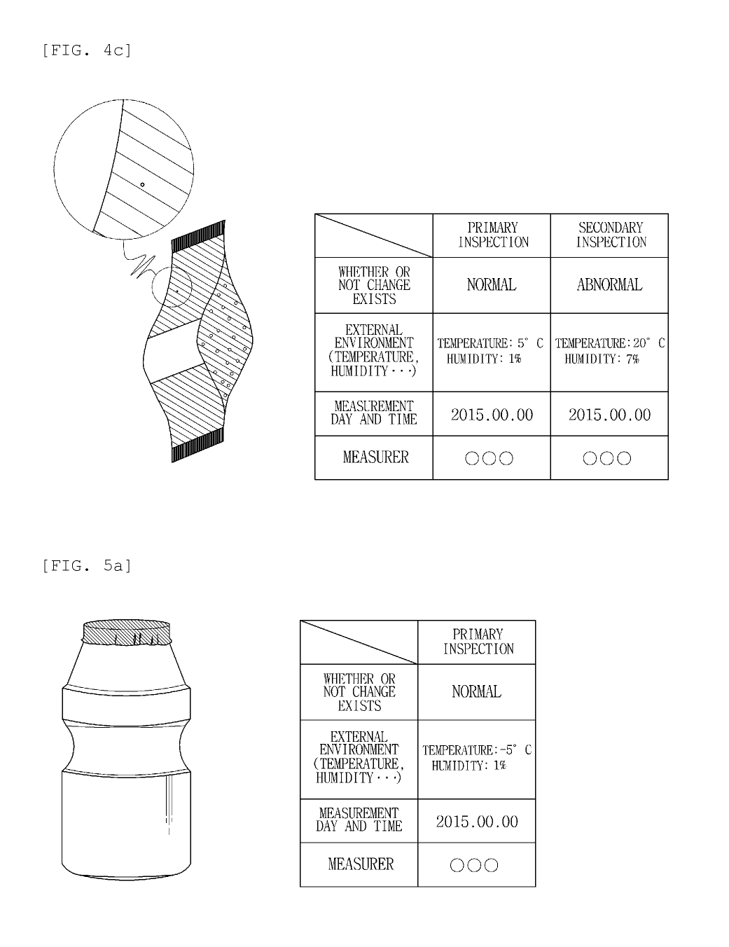

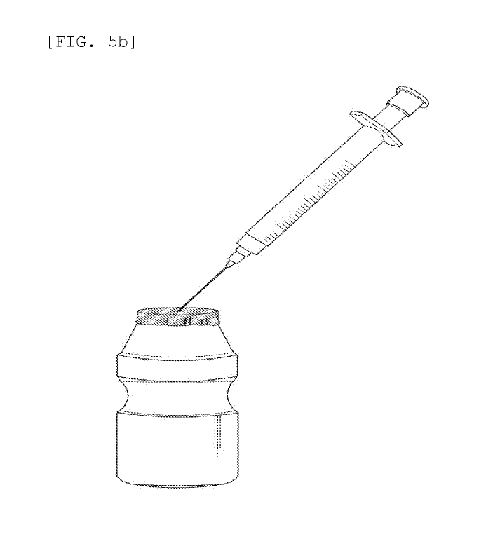

FIGS. 5A to 5C are views for describing a driving example of a package damage inspection device according to another exemplary embodiment of the present invention.

Referring to FIG. 5A, the package damage inspection device may obtain information in a manufacturing step. For example, the information may include information on whether or not the sealed container is maintained in the sealed state (‘information on whether or not the change exists’), internal environmental change information (a humidity, a temperature, a specific gas concentration, and the like), information on a measurement day and time, and information on a measurer. In the case in which the sealed container is in a normal state, a temperature may be −5° C. and a humidity may be 1%.

Referring to FIG. 5B, a criminal may inject a harmful material into the sealed container using a syringe in a distributing step, a selling step, or the like. As described above, a portion damaged by the syringe is very fine, such that it may not be determined whether or not the sealed container is damaged with the naked eyes.

Referring to FIG. 5C, the user, or the like, may inspect the sealed container using the package damage inspection device after the manufacturing step. In the case in which a temperature is 3° C. and a humidity is 8% as an inspection result, the package damage inspection device may determine that the sealed container is damaged from the sealing state since a difference of internal environmental change information is large as compared with the normal state (‘temperature: −5° C. and humidity: 1%’).

When sealing is finely damaged as in the present exemplary embodiment, entry of an external gas, liquid, or solid is allowed, such that an internal environment of the sealed container tends to become similar to an external environment.

Even in the case in which the sealing is finely damaged by the syringe as described above, when the package damage inspection device according to the present invention is used, it may be easily determined whether or not the sealed container is maintained in the sealed state.

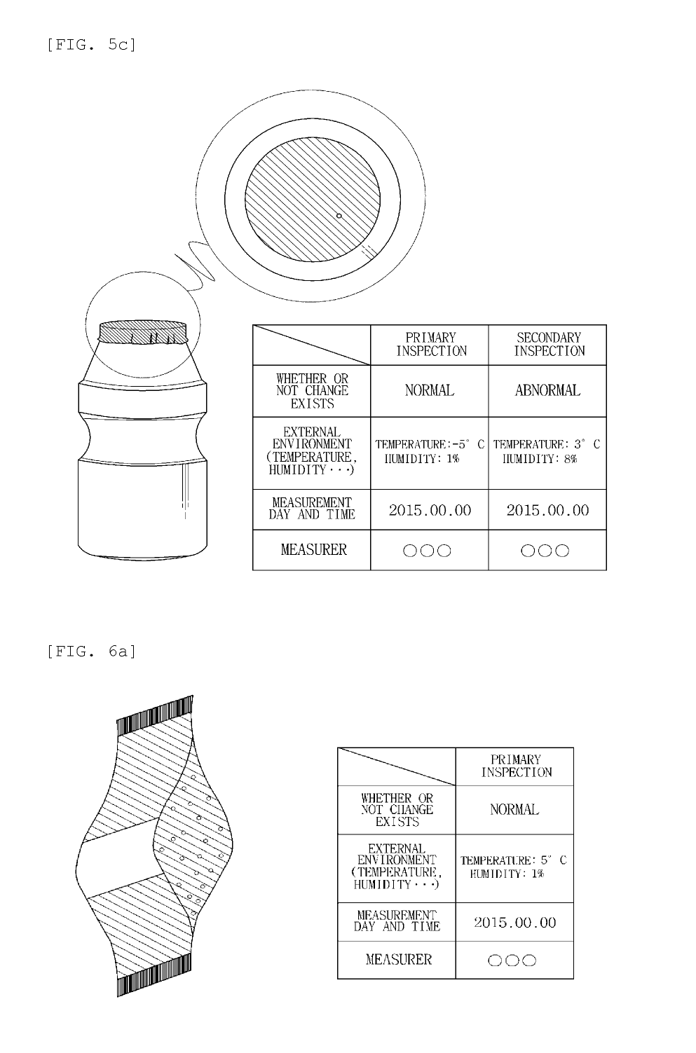

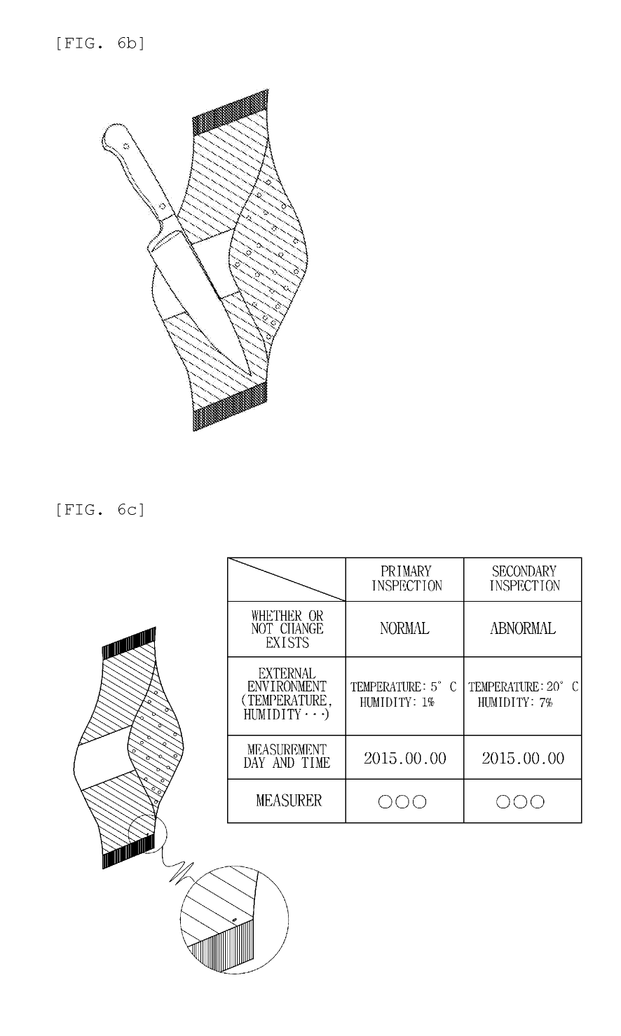

FIGS. 6A to 6C are views for describing a driving example of a package damage inspection device according to still another exemplary embodiment of the present invention.

Referring to FIG. 6A, the package damage inspection device may obtain information in a manufacturing step. For example, the information may include information on whether or not the sealed container is maintained in the sealed state (‘information on whether or not the change exists’), internal environmental change information (a humidity, a temperature, a specific gas concentration, and the like), information on a measurement day and time, and information on a measurer. In the case in which the sealed container is in a normal state, a temperature may be 5° C. and a humidity may be 1%.

Referring to FIG. 6B, the sealed container may be damaged by a sharp tool or be damaged due to drop in a process of carrying things, in a distributing step, a selling step, or the like. As described above, a portion damaged by the sharp tool or the drop is very fine, such that it may not be determined whether or not the sealed container is damaged with the naked eyes.

Referring to FIG. 6C, the user, or the like, may inspect the sealed container using the package damage inspection device after the manufacturing step. In the case in which a temperature is 20° C. and a humidity is 7% as an inspection result, the package damage inspection device may determine that the sealed container is damaged from the sealed state since a difference of internal environmental change information is large as compared with the normal state (‘temperature: 5° C. and humidity: 1%’).

Even in the case in which the sealing is finely damaged by the sharp tool as described above, when the package damage inspection device according to the present invention is used, it may be easily determined whether or not the sealed container is maintained in the sealed state.

FIG. 7 is a view for describing a container according to an exemplary embodiment of the present invention.

Referring to FIG. 7, a package container 700 including a region through which an electromagnetic wave is transmitted may include a space surrounded by a container 701 for an electromagnetic wave. A material such as a food, or the like, may be inserted into the space.

The container 701 for an electromagnetic wave may include a first electromagnetic wave transmitting layer 702, an electric field enhancing structure 703, a selective sensing layer 704, a filter layer 705, and an electromagnetic wave blocking layer 706.

The container 701 for an electromagnetic wave may include the electromagnetic wave transmitting layer 702 through which the electromagnetic wave may be transmitted and the electromagnetic wave blocking layer 706 by which the electromagnetic wave is blocked, and shapes and sizes of regions of the electromagnetic wave transmitting layer 702 and the electromagnetic wave blocking layer 706 may be variously modified. As described above, a region through which the electromagnetic wave is transmitted may be formed in only a portion of the package container 700 rather than the entirety of the package container 700. For example, the electromagnetic wave may be a terahertz wave.

The electromagnetic wave transmitting layer 702 may be formed of a material that may transmit the electromagnetic wave therethrough.

The electric field enhancing structure 703 may react to a preset frequency band in the electromagnetic wave transmitted through the electromagnetic wave transmitting layer 702 to enhance an electric field. For example, the electric field enhancing structure 703 may have various structures that may enhance the electric field, such as a diffraction grating, a metal mesh, a meta material, a metal layer including an opening having a width equal to or smaller than a wavelength of an electromagnetic wave generating unit, a structure inducing surface plasmon resonance, a photonic crystal structure, and the like.

The selective sensing layer 704 may be a layer in which a sensing material bonded to only a specific material is fixed to a support. For example, in the case in which the specific material is a specific ion, a specific gas, moisture, a harmful material, or the like, the selective sensing layer 704 may be bonded to only the specific ion, the specific gas, the moisture, or the harmful material, and may not be bonded to other materials.

The filter layer 705 may allow only the specific material to pass to the selective sensing layer 704. For example, the filter layer 705 may be formed at the innermost portion of the package container 700, and may allow only the specific material (for example, the specific ion, the specific gas, the moisture, or the harmful material) of various kinds of materials existing in an internal space of the package container 700 to pass to the selective sensing layer 704.

The electromagnetic wave blocking layer 706 may be formed at both sides of the terahertz wave transmitting layer 702, the electric field enhancing structure 703, the selective sensing layer 704, and the filter layer 705, and may reflect the electromagnetic wave.

The electromagnetic wave blocking layer 706, which is originally formed by coating a metal layer such as an aluminum layer on a polymer package material (polyethylene (PE) or polypropylene (PP)) in order to protect a product from an ultraviolet ray, a visible ray, an infrared ray, moisture, a harmful material, and the like, introduced from the outside of a package into the package, contains a metal component to reflect the electromagnetic wave.

In order to easily detect an inner portion of the container by a non-destructive method, a sensing window consisting of the electromagnetic wave transmitting layer 702, the electric field enhancing structure 703, the selective sensing layer 704, and the filter layer 705 may be formed in only a specific portion of the entire container.

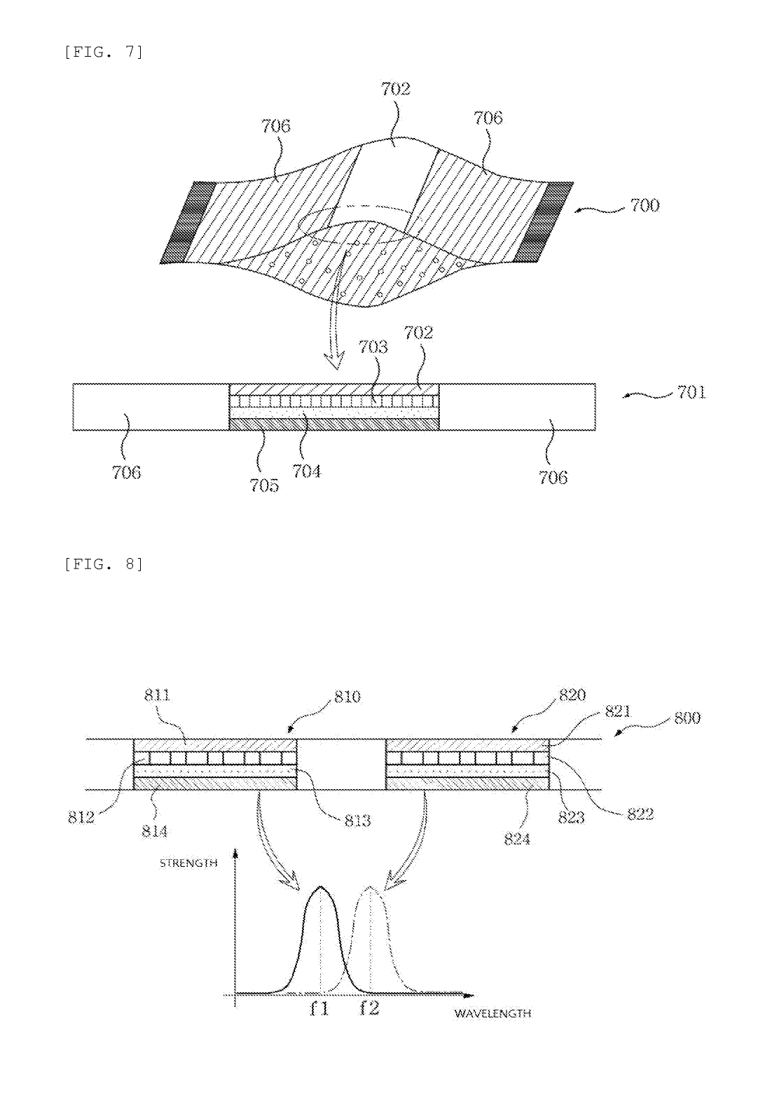

FIG. 8 is a view for describing a container according to another exemplary embodiment of the present invention.

Referring to FIG. 8, the container 800 may include a first region 810 that may obtain reference electromagnetic wave characteristics and a second region 820 that may obtain changed electromagnetic wave characteristics.

The first region 810 may include a terahertz wave transmitting layer 811, an electric field enhancing structure 812, a selective sensing layer 813 that does not include a sensing material, and a filter layer 814.

The second region 820 may include a terahertz wave transmitting layer 821, an electric field enhancing structure 822, a selective sensing layer 823 that includes a sensing material, and a filter layer 824.

Since functions of layers included in the respective regions are described above, a description therefor will be omitted.

When an electromagnetic wave generating unit (not illustrated) irradiates an electromagnetic wave to the first region 810, a detecting unit (not illustrated) may detect a first resonance frequency f1 of the electromagnetic wave detected in the first region 810. Here, the first resonance frequency f1 is a resonance frequency of a reference electromagnetic wave. When the electromagnetic wave generating unit (not illustrated) irradiates an electromagnetic wave to the second region 820, the detecting unit (not illustrated) may detect a second resonance frequency f2 of the electromagnetic wave detected in the second region 820. Here, the second resonance frequency f2 is a resonance frequency of an electromagnetic wave changed due to bonding between the sensing material included in the selective sensing layer 823 and a specific material. In other words, when the specific material is bonded to the selective sensing layer 823, the second resonance frequency f2 is changed.

A determining unit (not illustrated) may compare the first resonance frequency f1 (‘a resonance frequency of a reference terahertz wave’) of the electromagnetic wave detected in the first region 810 and the second resonance frequency f2 of the electromagnetic wave detected in the second region 820 with each other, and determine that physical, chemical, and biological changes are generated in a package container (not illustrated) when a difference between the first and second resonance frequencies is greater than a set range.

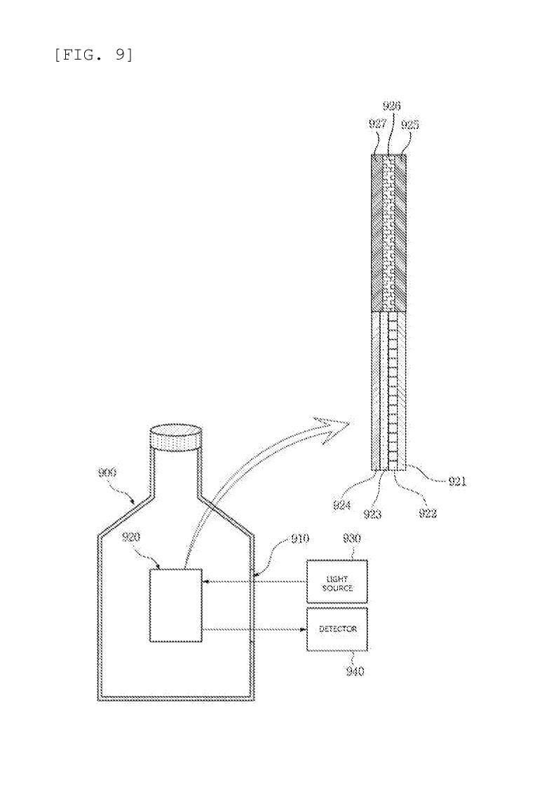

FIG. 9 is a view for describing a sensor included in the container according to an exemplary embodiment of the present invention.

Referring to FIG. 9, a package container 900 including a region through which an electromagnetic wave is transmitted may be a container in which a drink is contained. The package container 900 may include a region 910 through which the electromagnetic wave is transmitted. The region 910 may be formed in a portion of a side surface of the package container 900.

The sensor 920 may be provided in the package container 900.

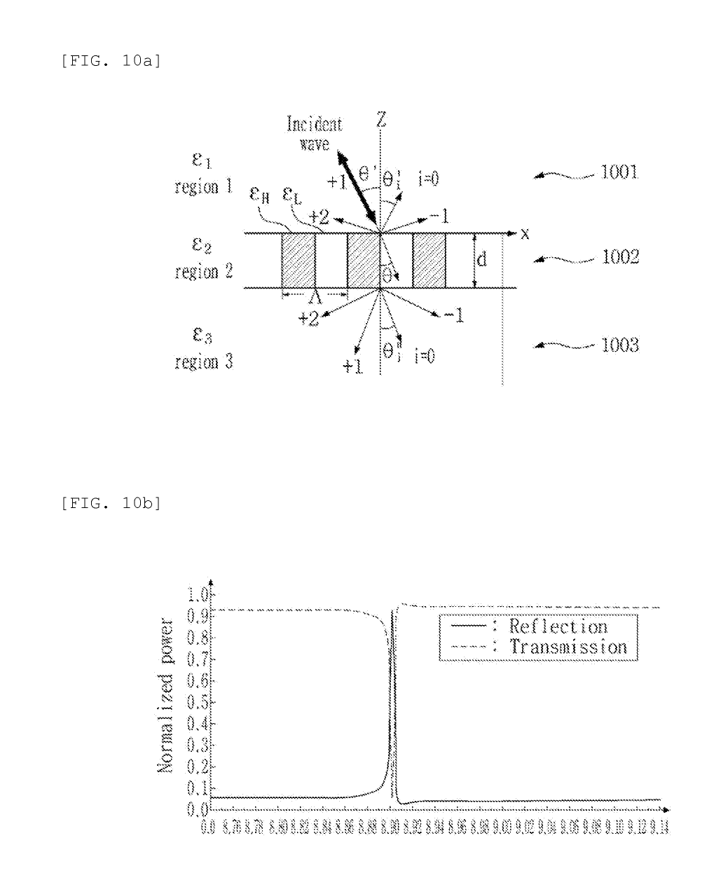

The sensor 920 may include a substrate layer 921, an electric field enhancing structure 922, a selective sensing layer 923, a filter layer 924, an electromagnetic wave transmitting layer 925, a waveguide diffraction grating 926, and a substrate layer 927. The substrate layer 921, the electric field enhancing structure 922, the selective sensing layer 923, the filter layer 924, and the electromagnetic wave transmitting layer 925 are components configured in order to sense an internal environmental change of the package, and the electromagnetic wave transmitting layer 925, the waveguide diffraction grating 926, and the substrate layer 927 are components (‘an optical identification element’) that may store an identification code given to the package. The substrate layer 921 and the electromagnetic wave transmitting layer 925 may be implemented in an integral form as one layer. The optical identification element will be described in detail below with reference to FIGS. 10 to 12C.

The substrate layer 921 may be formed of a material that may transmit the electromagnetic wave therethrough.

The electric field enhancing structure 922 may react to a preset frequency band in the electromagnetic wave transmitted through the substrate layer 921 to enhance an electric field. For example, the electric field enhancing structure 922 may have various structures that may enhance the electric field, such as a diffraction grating, a metal mesh, a meta material, a metal layer including an opening having a width equal to or smaller than a wavelength of an electromagnetic wave generating unit, a structure inducing surface plasmon resonance, a photonic crystal structure, and the like.

The selective sensing layer 923 may be a layer in which a sensing material bonded to only a specific material is fixed to a support. For example, in the case in which the specific material is a specific ion, a specific gas, moisture, a harmful material, or the like, the selective sensing layer 923 may be bonded to only the specific ion, the specific gas, the moisture, or the harmful material, and may not be bonded to other materials.

The filter layer 924 may allow only the specific material to pass to the selective sensing layer 923. For example, the filter layer 924 may be formed at the innermost portion of the package container 900, and may allow only the specific material (for example, the specific ion, the specific gas, the moisture, or the harmful material) of various kinds of materials existing in an internal space of the package container 900 to pass to the selective sensing layer 923.

In the case in which a change in moisture in the package container 900 is to be detected, a layer that may be bonded to only the moisture may be used as the selective sensing layer 923, and a layer that may allow only the moisture to pass therethrough may be used as the filter layer 924. For example, when the electromagnetic wave generating unit 930 irradiates the electromagnetic wave to the sensor 920, the detecting unit 940 may detect a resonance frequency of the electromagnetic wave sensed from the sensor 920. A determining unit (not illustrated) may compare the resonance frequency of the electromagnetic wave sensed from the sensor 20 and a resonance frequency (‘a resonance frequency in the case in which the moisture does not exist’) of a reference electromagnetic wave with each other, and determine that the moisture is generated in the vicinity of the electric field enhancing structure when a difference between the two resonance frequencies is greater than a set range. That is, the determining unit (not illustrated) may determine that the moisture is generated in the package container 900.

FIGS. 10A to 10C are views for describing an electric field enhancing structure according to an exemplary embodiment of the present invention.

Referring to FIG. 10A, the electric field enhancing structure may be a waveguide diffraction grating generating guided mode resonance (GMR) with respect to a specific wavelength.

A waveguide diffraction grating layer 1002 may diffract light incident thereto in given conditions (a wavelength and an incident angle of incident light, a thickness and an effective refractive index of a waveguide, and the like). High-order diffracted waves except for 0-order may form a guided mode in the waveguide diffraction grating layer 1002. In this case, 0-order reflected wave-transmitted wave are phase-matched to the guide mode, and resonance that energy of the guided mode is again transferred to the 0-order reflected wave-transmitted wave is generated. When the resonance is generated, a 0-order reflected diffracted wave is reflected 100% by constructive interference and a 0-order transmitted diffracted wave is transmitted 0% by destructive interference, resulting in drawing a very sharp resonance curve in a specific wavelength band.

FIG. 10B illustrates a GMR calculation result (resonance is generated at 0.89 THz) calculated by a finite difference element method in a case of forming a diffraction grating (n_{H}=1.80, n_{L}=1.72, thickness=80μ, and period=200 μm) using an SU-8 photoresist on a transparent polymethylpentene substrate (n=1.46) in an electromagnetic wave band.

As illustrated in FIG. 10A, when a permittivity of a cover layer 901 is ε₁, a permittivity of the waveguide diffraction grating layer 1002 is ε₂, and a permittivity of the lowermost substrate layer 1003 is ε₃, the permittivity (ε₂) of the waveguide diffraction grating layer 1002 may be represented by the following Equation 1:

ε₂(X)=ε_{g}+Δε*cos(Kx). [Equation 1]

Here, ε_{g} is an average value of two kinds of permittivities ε_{H} and ε_{L} constituting the diffraction grating and repeated, Δε is a maximum change amount in a permittivity, K, which is a wave number of the grating, is 2π/Λ, Λ is a period of the grating, x is a distance from a starting point in an X-axis direction.

In this case, in order to generate resonance, that is, a guided mode, in the waveguide diffraction grating at a specific wavelength and incident angle of incident light, an effective refractive index (N) of the waveguide needs to satisfy the following condition:

max(sqrt(ε₁,ε₃)|N|<sqrt(ε_{g}).

When the GMR is generated in the waveguide diffraction grating, a phenomenon in which an electric field is concentrated near the waveguide diffraction grating is well known, and a fine change in a refractive index near the diffraction grating entirely appears as a change in a resonance frequency due to such a near field enhancement phenomenon. Such a principle may be utilized as a high sensitivity sensing principle since chemical-physical bonding of a fine sensing material generated in a sensing film formed near the waveguide diffraction grating appears as the change in the resonance frequency.

Here, such a principle is applied in an electromagnetic wave region to form a GMR sensing element reacting in the electromagnetic wave region in the container, thereby making it possible to manufacture an electromagnetic wave sensing element at a high sensitivity. Particularly, non-destructive detection is possible at a high sensitivity by combination with non-destructive characteristics of the electromagnetic wave.

FIG. 10C is a perspective view for describing a structure and a shape of the waveguide diffraction grating.

The waveguide diffraction grating may include grooves or ridges formed on a surface of a dielectric slab. As another example, the waveguide diffraction grating is a planar dielectric sheet having periodically alternating refractive indices (for example, phase gratings) therein. Such phase gratings may be formed by forming an array of periodical holes passing through the dielectric sheet in the dielectric sheet.

As still another example, the waveguide diffraction grating may include any one of a one-dimensional (1D) diffraction grating and a two-dimensional (2D) diffraction grating. The 1D diffraction grating may include, for example, a set of grooves, which are substantially straight lines periodical and parallel with each other in only a first direction (for example, along an x axis). An example of the 2D diffraction grating may include an array of holes in a dielectric slab or sheet. Here, the holes are periodically spaced apart from each other in two orthogonal directions (for example, along x and y axes). Here, the 2D diffraction grating is also called a photonic crystal.

FIG. 11 is a view for describing an optical identification element according to an exemplary embodiment of the present invention.

Referring to FIG. 11, the optical identification element 1100 may include m identification units. Each of the identification units may include an electromagnetic wave transmission layer 1110, a waveguide diffraction grating 1120, and a substrate layer 1130. Although a case in which the number of identification units is eight will be described in the present exemplary embodiment, the number of identification units is not limited thereto. An area of the identification unit may be affected by an irradiation area, a natural resonant frequency, a grating period, and the like, and may be largest affected by the irradiation area among them. For example, in the case in which a diameter of an irradiation beam of an electromagnetic wave is 6 mm, an area of the identification unit may be 8 mm*8 mm. As described above, the diameter of the irradiation beam of the electromagnetic wave is small, and the area of the identification unit is thus very small. The optical identification element 112 of FIG. 1 may be implemented by the optical identification element 1100 according to the present exemplary embodiment.

The electromagnetic wave transmitting layer 1110 may be formed of a material that may transmit the electromagnetic wave therethrough.

The waveguide diffraction grating 1120 may generate an electromagnetic wave having a natural resonant frequency when an electromagnetic wave transmitted through the electromagnetic wave transmitting layer 1110 is irradiated thereto. Here, the natural resonant frequency may be any one of a first natural resonant frequency to an n-th natural resonant frequency. For example, the first natural resonant frequency may be f₁. When n is 10, the natural resonant frequency may be any one of ten unique resonance frequencies.

The waveguide diffraction grating 1120 may be formed of a material such as a photosensitive material, a heat-sensitive material, an electro-active material, and the like.

The waveguide diffraction grating 1120 may include grooves or ridges formed on a surface of a dielectric slab. As another example, the waveguide diffraction grating is a planar dielectric sheet having periodically alternating refractive indices (for example, phase gratings) therein. Such phase gratings may be formed by forming an array of periodical holes passing through the dielectric sheet in the dielectric sheet.

The waveguide diffraction grating 1120 may include any one of a 1D diffraction grating and a 2D diffraction grating. The 1D diffraction grating may include, for example, a set of grooves, which are substantially straight lines periodical and parallel with each other in only a first direction (for example, along an x axis). An example of the 2D diffraction grating may include an array of holes in a dielectric slab or sheet. Here, the holes are periodically spaced apart from each other in two orthogonal directions (for example, along x and y axes). Here, the 2D diffraction grating is also called a photonic crystal.

The substrate layer 1130 may be a layer that may be coupled to the waveguide diffraction grating 1120 to fix the waveguide diffraction grating 1120.

When a kind of unique resonance frequencies is n and the number of identification units is m, the number of identification codes that may be represented by the optical identification element 1100 is n^{m}. As an example, in the case in which a kind of unique resonance frequencies is 10 and the number of identification units is 2, the number of identification codes is 10²=100. As described above, the optical identification element 1100 may represent 100 identification codes in spite of using only two identification units. As another example, in the case in which a kind of unique resonance frequencies is 10 and the number of identification units is 8, the number of identification codes is 10⁸=100,000,000.

Therefore, the optical identification element 1100 may represent a large number of identification codes within a small area.

In addition, the optical identification element may not be recognized with the naked eyes, and security of the optical identification element is thus excellent.

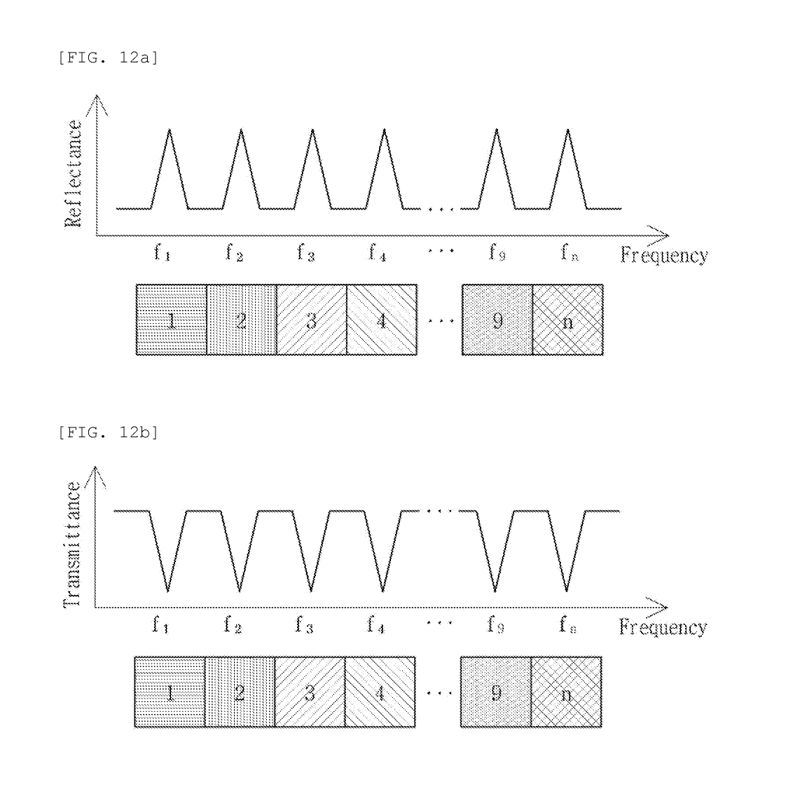

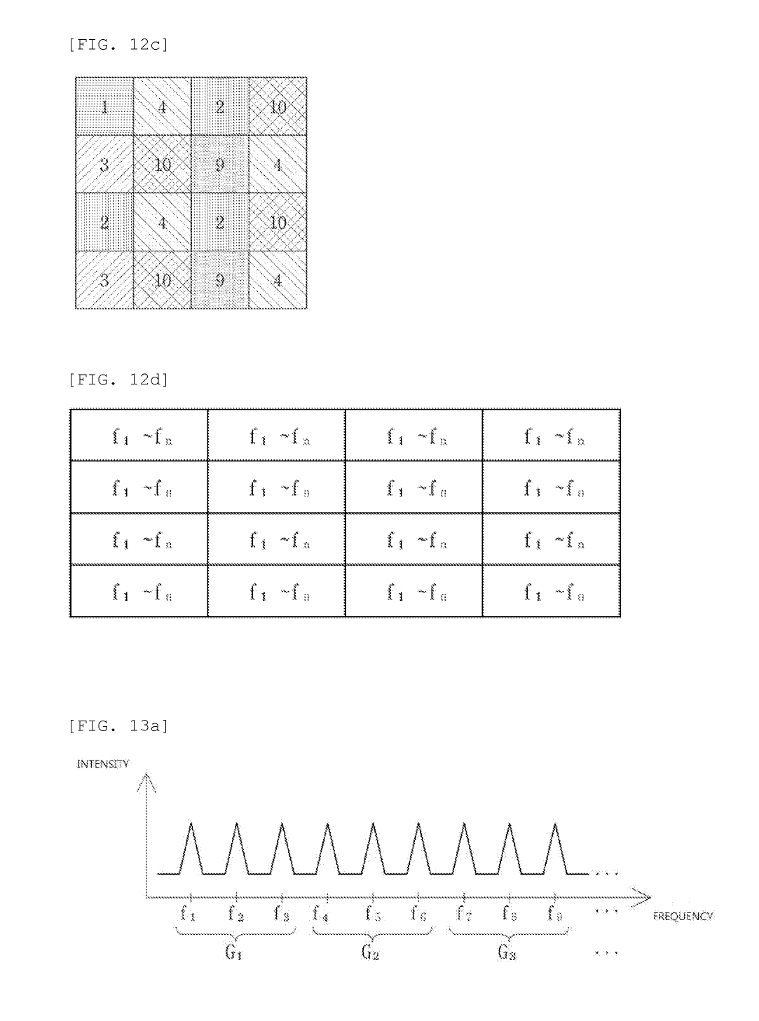

FIGS. 12A to 12D are views for describing the optical identification element according to an exemplary embodiment of the present invention in detail.

FIG. 12A is a graph illustrating a detection result of an electromagnetic wave reflected from the optical identification element.

Referring to FIG. 12A, the respective identification units 1 to n may have unique resonance frequencies f₁, f₂, f₃ to fₙ, respectively. For example, a first identification unit 1 may have a first natural resonant frequency f₁, a second identification unit 2 may have a second natural resonant frequency f₂, and an n-th identification unit may have an n-th natural resonant frequency fₙ.

FIG. 12B is a graph illustrating a detection result of an electromagnetic wave transmitted through the optical identification element.

Referring to FIG. 12B, the respective identification units 1 to n may have unique resonance frequencies f₁, f₂, f₃ to fₙ, respectively. For example, a first identification unit 1 may have a first natural resonant frequency f₁, a second identification unit 2 may have a second natural resonant frequency f₂, and an n-th identification unit may have an n-th natural resonant frequency fₙ.

FIG. 12C is a view for describing an optical identification element including sixteen identification units.

Referring to FIG. 12C, the total number of identification units is sixteen, and a total of sixteen identification units may be formed of combinations of ten identification units 1 to 10 having the respective unique resonance frequencies f₁, f₂, f₃ to f₁₀. In detail, a 1st identification unit may be a first identification unit 1 having a first natural resonant frequency f₁, a 2nd identification unit may be a fourth identification unit 4 having a fourth natural resonant frequency f₄, a 3rd identification unit may be a second identification unit 2 having a second natural resonant frequency f₂, and identification units existing at the other positions may be formed of identification units, as illustrated in FIG. 2C.

FIG. 12D is a view for describing the number of identification codes that may be represented in the case in which a kind of unique resonance frequencies is n and the number of identification units is m.

Referring to FIG. 12D, since a kind of unique resonance frequencies of identification units that may be formed in the respective identification units is n and the optical identification elements include a total of sixteen identification units, the number of identification codes that may be represented may be n¹⁶.

FIG. 12E is a view for describing various forms of arrangements of identification units.

Referring to FIG. 12E, the identification units may be arranged in various forms, and forms of arrangements may mean identification information different from identification codes. The identification units may be arranged in various forms such as a linear form, a circular form, a rectangular form, a grating form, a cross form, and the like.

Referring to (a) to (c) of FIG. 12E, the identification units may be arranged in a linear form, a cross form, and a circular band form. Here, the linear form may mean thing A, the cross form may mean thing B, and the circular band form may mean thing C. As described above, the forms in which the identification units are arranged may also be used as identification information.

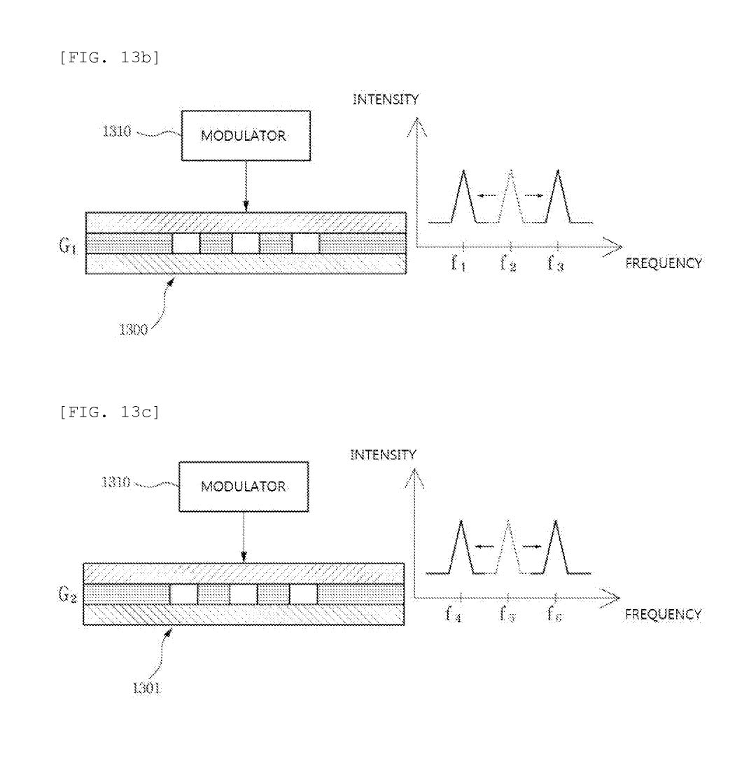

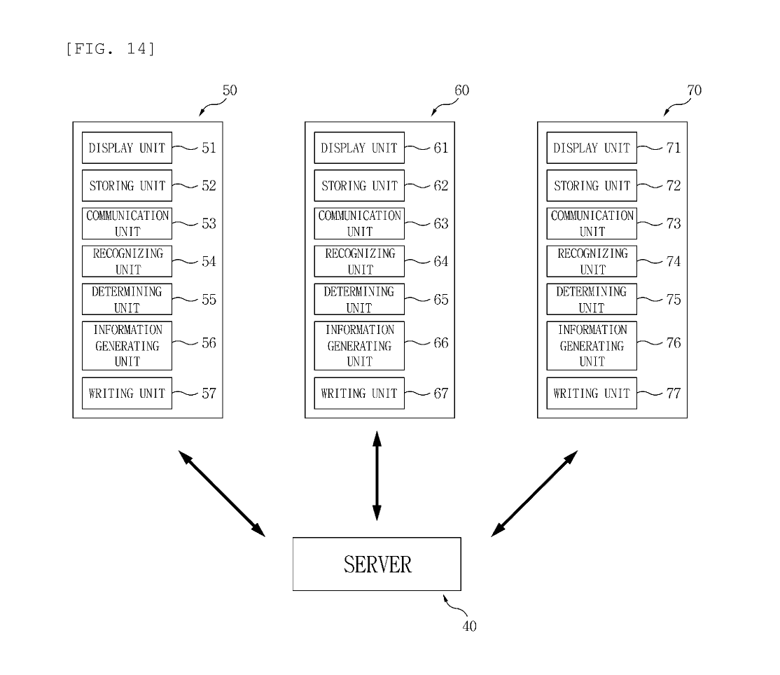

FIGS. 13A to 13C are views for describing a writing device for an identification unit according to an exemplary embodiment of the present invention.

Referring to FIG. 13A, unique resonance frequencies may be set for each of frequency bands G1, G2, . . . , Gm. The frequency bands may be set on the basis of frequency bands that may be changed by a modulation unit 1310b (see FIG. 13B). For example, a frequency band that may be changed by the modulation unit 1310b (see FIG. 13B) on the basis of f₂ is f₁ to f₃, a first frequency band G1 may be f₁ to f₃. A frequency band that may be changed by the modulation unit 1310b (see FIG. 13B) on the basis of f₅ is f₄ to f₆, a first frequency band G1 may be f₄ to f₆.

Referring to FIG. 13B, a writing device for an identification unit may include an identification unit 1300b and the modulating unit 1310b. The identification unit 1300b may include a terahertz wave transmitting layer formed of a material transmitting a terahertz wave therethrough and a waveguide diffraction grating having a natural resonant frequency f₂ corresponding to the frequency band G1 set with respect to the transmitted terahertz wave.

The modulating unit 1310b may change the natural resonant frequency of the waveguide diffraction grating into another natural resonant frequency within the set frequency band. For example, the modulating unit 1310b may change the natural resonant frequency f₂ of the waveguide diffraction grating into another natural resonant frequency f₁ or f₃ within the set frequency band G1.

As a specific example for a method of changing the resonance frequency, the modulating unit 1310b may change the natural resonant frequency of the waveguide diffraction grating into another natural resonant frequency within the set frequency band.

Referring to FIG. 13C, a writing device for an identification unit may include an identification unit 1300c and the modulating unit 1310c. The identification unit 1300c may include a terahertz wave transmitting layer formed of a material transmitting a terahertz wave therethrough and a waveguide diffraction grating having a natural resonant frequency f₅ corresponding to the frequency band G2 set with respect to the transmitted terahertz wave.

The modulating unit 1310c may change the natural resonant frequency of the waveguide diffraction grating into another natural resonant frequency within the set frequency band. The modulating unit 1310c may change the natural resonant frequency f₅ of the waveguide diffraction grating into another natural resonant frequency f₄ or f₆ within the set frequency band G2.

As described above, when the writing device for an identification unit is used, a user, or the like, may freely change the resonance frequency of the identification unit within the set resonance frequency range. Therefore, the identification units do not need to be produced for each resonance frequency, and production costs of the identification units and the optical identification element may thus be reduced. In addition, the user, or the like, may change the resonance frequency of the identification unit into a desired resonance frequency in the field using the writing device for an identification unit, thereby making it possible to increase convenience of the user.

FIG. 14 is a view for describing a package damage inspection system according to an exemplary embodiment of the present invention.

Referring to FIG. 14, the package damage inspection system may include one or more package damage inspection devices 50, 60, and 70, and a server 40.

A plurality of package damage inspection devices 50, 60, and 70 may exist, and the respective package damage inspection devices 50, 60, and 70 may be a device used in a manufacturing step, a device used in a distributing step, and a device used in a selling step, and the like.

The package damage inspection devices 50, 60, and 70 may include sensors 51, 61, and 71 recognizing internal environmental change information of a sealed container, identification elements (not illustrated) including identification codes of the sealed container, recognizing units 54, 64, and 74 recognizing the identification codes included in the identification elements to recognize identification information of the container, and determining units 55, 65, and 75 comparing the internal environmental change information recognized by the sensors and reference change information with each other to determine whether or not the sealed container is maintained in a sealed state, respectively.

Since the respective components included in the package damage inspection devices 50, 60, and 70 are described above, a description therefor will be omitted in the present exemplary embodiment.

The server 40 may receive information on whether or not the sealed container is maintained in the sealed state from the package damage inspection devices 50, 60, and 70, and store the received information on whether or not the sealed container is maintained in the sealed state in a storing unit or transmit the received information on whether or not the sealed container is maintained in the sealed state to a terminal of a manager.

The package damage inspection devices 50, 60, and 70 may be provided in each of a first distributing step, a second distributing step, and an N-th distributing step. In this case, the server 40 may receive the information on whether or not the sealed container is maintained in the sealed state, internal environmental change information for each identification code and each distributing step, external environment information at the time of performing measurement, information on a measurement day and time, and information on a measurer from the package damage inspection devices in each of the first distributing step, the second distributing step, and the N-th distributing step.

The determining units 55, 65, and 75 may compare the internal environmental change information recognized by the sensors and reference change information corresponding to the identification codes of the sealed container and corresponding to a current distributing step with each other to determine whether or not the sealed container is maintained in the sealed state.

The determining units 55, 65, and 75 may compare the internal environmental change information recognized by the sensors and the reference change information with each other to determine whether or not a change in an internal environment exists, and determine that the sealed container is not maintained in the sealed state in the case in which it is determined that the change in the internal environment of the sealed container exists.

The determining units 55, 65, and 75 may compare the internal environmental change information generated in the current measuring step and the internal environmental change information generated in the previous measuring step with each other to determine that the sealed container is not maintained in the sealed state.

The determining units 55, 65, and 75 may compare the internal environmental change information generated in the current measuring step and current external environment information with each other to determine whether or not the sealed container is maintained in the sealed state.

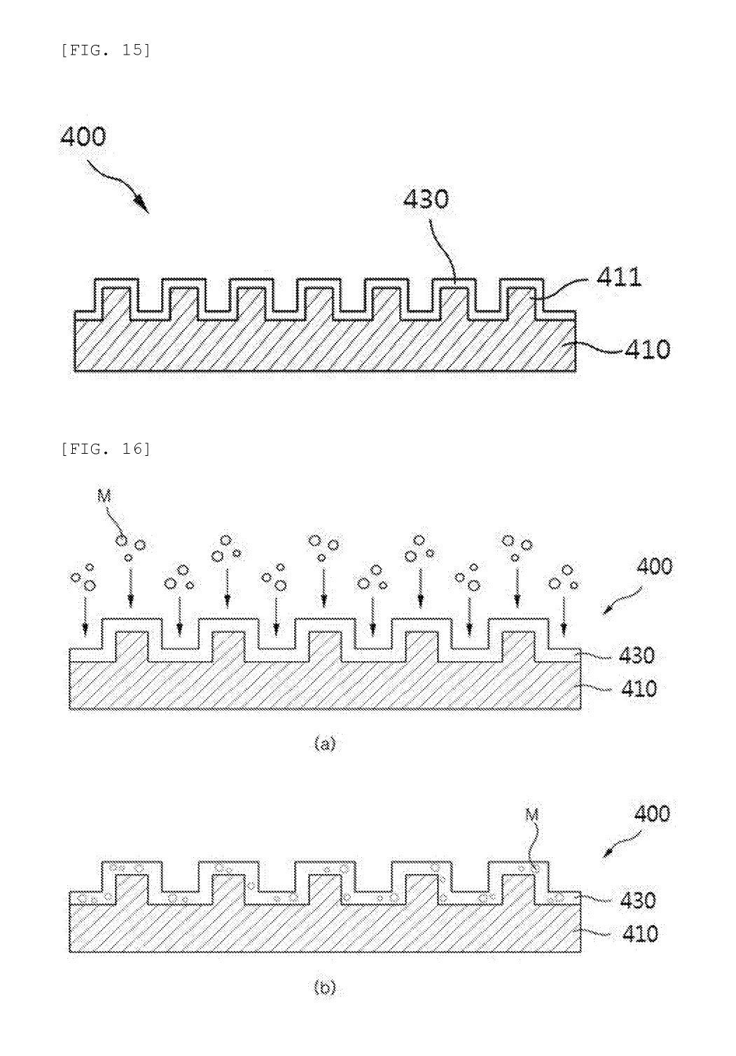

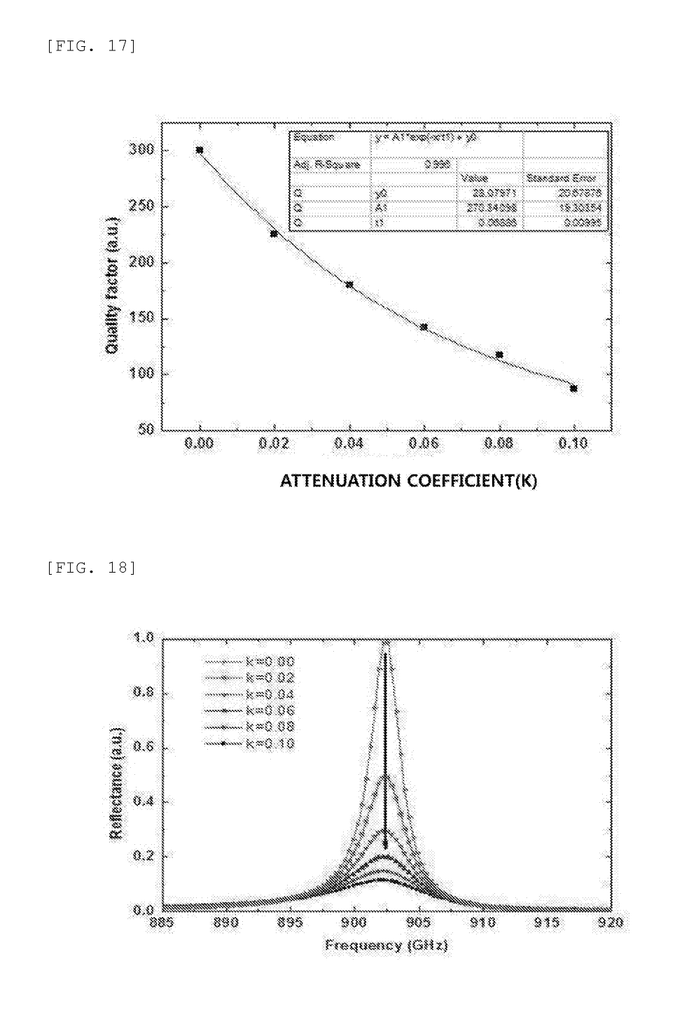

FIG. 15 is a side view for describing a structure of a humidify sensor 400 according to an exemplary embodiment of the present invention, and FIGS. 16A and 16B are views for describing an operation method of the humidify sensor 400 of FIG. 15.

As illustrated in FIG. 15, the humidity sensor 400 may include a guided mode resonance (GMR) element 410 and a moisture sensing film 430.

In the GMR element 440, after light incident to a diffraction grating is diffracted in given conditions (a wavelength and an incident angle of incident light, a thickness and an effective refractive index of a waveguide, and the like), high-order diffracted waves except for 0-order may be converted into a leaky guided mode formed in a waveguide diffraction grating. In this case, 0-order reflected wave-transmitted wave are phase-matched to the leaky guide mode, and resonance that energy of the leaky guided mode is again transferred to the 0-order reflected wave-transmitted wave is generated.

When the resonance is generated, a 0-order reflected diffracted wave is reflected 100% by constructive interference and a 0-order transmitted diffracted wave is transmitted 0% by destructive interference, resulting in an increase in a quality factor while drawing a very sharp resonance curve in a specific wavelength band. Therefore, in the case in which a first electromagnetic wave such as a terahertz wave is irradiated from the outside depending on the principle described above, the GMR element 140 may generate a second electromagnetic wave having a specific wavelength band and quality factor depending on a diffraction grating formed of a grating layer 411. In addition, environment information (humidity information) on a place in which the GMR element 410 is disposed may be sensed by an analysis of the second electromagnetic wave.

Here, the quality factor, which is an index used in order to represent performance of a general resonance structure, may be represented by a value obtained by dividing a resonance frequency (fr) by a full width half maximum frequency (Δf). Therefore, an equation for calculating the quality factor may be defined as Q=f/Δf. For example, in the case in which the second electromagnetic wave generated from the humidity sensor 400 has a resonance frequency of 900 GHz and a full width half maximum frequency of 50 GHz, a quality factor of 18 may be calculated depending on an Equation of Q=900/50.

The moisture sensing film 430 may be formed of a material that may absorb moisture, and may be applied onto the GMR element 410. In other words, the moisture sensing film 430 may be applied onto the grating layer 411 of the GMR element 410 to change a property of the second electromagnetic wave generated by irradiating the first electromagnetic wave to the GMR element 410.

The moisture sensing film 430 may include one or more selected from the group consisting of moisture adsorbing inorganic materials including lithium chloride, silica gel, and activated alumina or water adsorbing organic materials including a carboxyl group (—COOH), an amine group (—NH2), and an alcohol group (—OH).

The lithium chloride has deliquescence, such that it absorbs moisture in the air to be melted.

Since the silica gel has numerous holes corresponding to about 50% of a volume to have a large surface area, adsorptive power between water vapor and a gas is large.

Since the activated alumina is porous and has a large surface area, it may adsorb moisture and acid. The activated alumina has moisture absorption force higher than that of calcium chloride and the silica gel.

A low molecular or high molecular weight organic material having the carboxyl group, the amine group, and the alcohol group is adsorbed through hydrogen bond to moisture, and the functional groups described above may be formed by applying a material to the grating layer or performing physical treatment such as plasma treatment without applying the material.

Referring to FIGS. 16A and 16B, in the case in which moisture M exists in the vicinity of the GMR element 410, the moisture M may be adsorbed by the moisture sensing film 430. In this case, an attenuation coefficient of the moisture sensing film 130 may be changed, and characteristics (a wavelength band, a quality factor, and the like) of the second electromagnetic wave generated by the GMR element 410 may be changed depending on the changed attenuation coefficient. Therefore, the humidity sensor 400 using a principle that the second electromagnetic wave is changed depending on a level of the attenuation coefficient of the moisture sensing film 430 may be implemented.

Here, the attenuation coefficient may be a numerical value for a level in which light energy of the first electromagnetic wave irradiated toward the moisture sensing film 430 is absorbed in and attenuated by water molecules of the moisture sensing film 430 in the case in which moisture is adsorbed in the moisture sensing film 430. In other words, the attenuation coefficient, which is a coefficient indicating a ratio in which an electric wave, or the like, is attenuated when it passes through a specific material, may be calculated as μ from A=A₀*e^{-μx}, which is an equation for calculating a light flux or an electric wave intensity A. Here, x is a thickness of a material, A₀ is a value when x=0, A is a light flux or a intensity of an electric wave when the electric wave passes through x, and p is an attenuation coefficient.

In addition, the attenuation coefficient is an absorption coefficient of a complex refractive index. In detail, when light enters a conductive medium such as a metal, a conduction current that is in proportion to an electrical spectrum is generated, in addition to an electric flux current that is in proportion to a change in an electric vector over time. Since this current, which is an electric flux current, has a phase different from that of the electric flux current by about 90°, the conductive medium may be treated as a medium binding the two currents as one in consideration of the phases of the two currents to generate only the electric flux current, that is, a non-conductor medium. In this case, a permittivity of the conductive medium is ε0−i4πσ/ω, which is a complex number, and a refractive index defined as sqrt(ε*μ) is also a complex number. It is called a complex refractive index. Here, ε0 is a permittivity generating an original electric flux current, i4πσ/ω is a permittivity of the conduction current when being considered as an electric flux current in consideration of the phase, σ is a conductivity, ω is an angular frequency of light in the medium, i is an imaginary number unit, and μ is a magnetic permeability (the attenuation coefficient μ described above and the magnetic permeability μ of the complex refractive index, which are the same reference signs applied to different equations, are differently interpreted). Therefore, the complex refractive index is represented by

n=n₀−i*kₒ=n₀(1−i*k).

Here, n₀ may be a refractive index, kₒ may be an extinction coefficient, and k may be an absorption coefficient.

Therefore, in the case in which the first electromagnetic wave is irradiated toward the humidity sensor 400 according to the present invention, the second electromagnetic wave corresponding to the first electromagnetic wave may be generated through the moisture sensing film 430. The second electromagnetic wave is changed depending on the attenuation coefficient or the absorption coefficient of the moisture sensing film 430, the corresponding change level may be compared with a reference value to calculate humidity information of a target object.

The GMR element 410 having the moisture sensing film 430 has been described hereinabove. In FIGS. 17 and 18, a process in which a property of a second electromagnetic wave is changed depending on a change of a moisture sensing film 430 will be described in detail.

FIG. 17 is a view for describing a change in a quality factor of a second electromagnetic wave depending on a change in an attenuation coefficient of a moisture sensing film 430. (Although the attenuation coefficient is represented by μ in FIGS. 16A and 16B, this is only a representation of a related equation, and the attenuation coefficient will be called k for easiness of explanation in the present drawing.)

As illustrated, a calculation result (a finite difference element analysis) in which a quality factor (Q=f/Δf (here, f is a resonance frequency and Δf is a full width half maximum frequency) in FIG. 17) is decreased in the case in which the attenuation coefficient k of moisture of the moisture sensing film 130 for an absorption level is increased. In other words, the quality factor of the second electromagnetic wave may be changed while substantially maintaining a constant correlation with the attenuation coefficient of the moisture sensing film 430 depending on the attenuation coefficient of the moisture sensing film 430. In detail, in the case in which the attenuation coefficient is increased from 0 to 1.0 in a unit of 0.02, the quality factor is gradually decreased from about 300 to 80.

Therefore, in the case in which the GMR element 410 to which the moisture sensing film 430 is applied is installed in a target object and the second electromagnetic wave is generated from the GMR element 410 by irradiating the first electromagnetic wave to the GMR element 410, the quality factor of the second electromagnetic wave may be changed depending on the attenuation coefficient of the moisture sensing film 430, and the corresponding change ratio may be established through simulation data.

Since the GMR element 410 having the moisture sensing film 430 and the quality factor of the second electromagnetic wave have the correlation described above, the corresponding GMR element 410 may be used as the humidity sensor 400 that may be disposed in a container of a product to sense a change in a humidity in the container.

For example, a case in which a humidity is defined as A % when a quality factor is 300, a humidity is defined as B % when a quality factor is 200, and a humidity is defined as C % when a quality factor is 100 is assumed. A detecting unit measures an electromagnetic wave generated from the humidity sensor 400, and a humidity information generating unit calculates a quality factor on the basis of the measured electromagnetic wave. The humidity information generating unit may derive a corresponding humidity value (A %, B %, or C %) depending on the calculated quality factor to generate humidity information. Therefore, the device according to the present invention may measure the humidity value depending on the change in the quality factor.

FIG. 18 is a view for describing a change in a reflectance of a second electromagnetic wave depending on a change in an attenuation coefficient of the moisture sensing film 430.

As illustrated, in the humidity sensor including the moisture sensing film, a reflectance as well as the quality factor in FIG. 17 is changed. The reflectance, which is a value indicating a level in which the first electromagnetic wave is irradiated and is then reflected by the humidity sensor, may be calculated as a detection value of the second electromagnetic wave.

In detail, in the case in which the attenuation coefficient of the moisture sensing film 430 is increased from 0 to 0.1 by a unit of 0.02 as in FIG. 17, the reflectance is decreased from 1 to about 0.1.

According to an experiment result as described above, it may be appreciated that a correlation (a decrease in the reflectance at the time of an increase in the attenuation coefficient) exists between the attenuation coefficient and the reflectance. Therefore, in the case in which the reflectance of the second electromagnetic wave generated from the humidity sensor is measured and is compared with a reference value, humidity information of the humidity sensor may be measured.

For example, a case in which a humidity is defined as A % when a reflectance is 1, a humidity is defined as B % when a reflectance is 0.5, and a humidity is defined as C % when a reflectance is 0.2 when a quality factor is 100 is assumed. A detecting unit measures an electromagnetic wave generated from the humidity sensor 400, and a humidity information generating unit calculates a reflectance on the basis of the measured electromagnetic wave. The humidity information generating unit may derive a corresponding humidity value (A %, B %, or C %) depending on the calculated reflectance to generate humidity information. Therefore, the device according to the present invention may measure the humidity value depending on the change in the reflectance.

Furthermore, since both of the quality factor of FIG. 17 and the reflectance of FIG. 18 tend to be decreased in the case in which the attenuation coefficient is increased, both of the quality factor and the reflectance of the second electromagnetic wave are calculated, and the humidity information is obtained through a combination of the calculated quality factor and reflectance, such that more accurate humidity information may be generated.

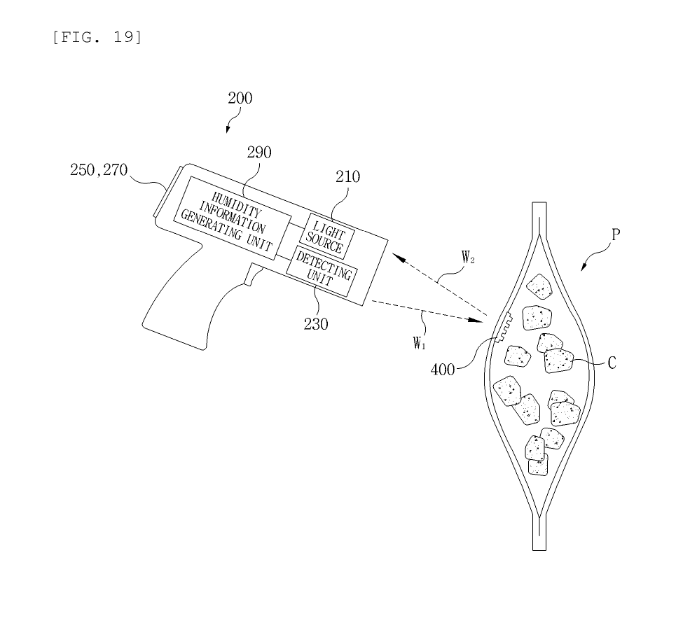

FIG. 19 is a view for describing a container inspection device 200 according to another exemplary embodiment of the present invention.

Referring to FIGS. 15 and 19, the container inspection device 200 may recognize humidity information in a container P by recognizing the humidity sensor 400 in the case in which the humidity sensor 400 of FIG. 15 is installed in the container P in which a content C is sealed. To this end, the container inspection device may include a light source 210, a detecting unit 230, a user input unit 250, a display unit 270, and a humidity information generating unit 290.

The light source 210 is a means for irradiating a first electromagnetic wave W₁ toward the humidity sensor 400. For example, the light source 210 may be various types of devices that may generate a terahertz wave. The terahertz wave, which is the first electromagnetic wave W₁ positioned in a region between an infrared ray and a microwave, may generally have a frequency of 0.1 THz to 10 THz. However, even though the terahertz wave is slightly out of the range described above, the terahertz wave may be considered as the terahertz wave in the present invention when it is in a range that may be easily deduced by those skilled in the art to which the present invention pertains.

The detecting unit 230 may detect a second electromagnetic wave W₂ generated from the humidity sensor 400. In other words, the detecting unit 230 may detect a intensity (a reflectance), a quality factor, and the like, of the second electromagnetic wave W₂ reflected from the humidity sensor 400.

The user input unit 250 may input component information, thickness information, and reflective index information of a moisture sensing film, frequency information of the first electromagnetic wave W₁, and the like. The moisture sensing film 430 used in the humidity sensor 400 and the frequency information of the first electromagnetic wave W₁ may be changed depending on a kind of a product container P, which is an inspection target. Information on a kind of the humidity sensor 100 installed in the corresponding container P may be input to and be changed by the user input unit 250.

The display unit 270 may visually output humidity information generated by a humidity information generating unit 290 to be described below and various information. Therefore, the user input unit 250 and the display unit 270 may be integrated with each other as a device such as a touch screen.

The humidity information generating unit 290 may generate the humidity information on the product container P on the basis of the second electromagnetic wave W₂ detected fro the detecting unit 230. In other words, the humidity information generating unit 290 may generate the humidity information corresponding to at least one of the reflectance and the quality factor of the second electromagnetic wave W₂ on the basis of at least one of the reflectance and the quality factor of the second electromagnetic wave W₂.

An operation method of the container inspection device 200 is as follows.

First, a user may input information (component information, thickness information, reflective index information, and the like, of the moisture sensing film 430) corresponding to a kind of humidity sensor 400 installed in the product container P that is to be inspected to the user input unit 250, and set frequency information of the first electromagnetic wave W₁ that is irradiated. When the corresponding setting is completed, the user may irradiate the first electromagnetic wave W₁ toward a region of the container P in which the humidity sensor 400 exists using the container inspection device 200. The first electromagnetic wave W₁ may react to the humidity sensor 400 to be converted into the corresponding second electromagnetic wave W₂. Here, in the case in which the moisture sensing film 430 adsorbs moistures, a property of the second electromagnetic wave W₂ may be changed due to the reason described above in FIGS. 16A and 16B, and the second electromagnetic wave W₂ of which the property is changed may be reflected to the detecting unit 230 of the container inspection device 200.

When the second electromagnetic wave W₂ is detected through the detecting unit 230 as described above, the humidity information generating unit 290 may compare a preset reference value and the detected second electromagnetic wave W₂ with each other. In detail, the humidity information generating unit 290 may compare the reflectance and the quality factor of the second electromagnetic wave W₂ with reference values to generate humidity information on whether or not the humidity sensor 400 contains moisture or an amount of contained moisture.

The generated humidity information may be displayed on the display unit 270 or be output as an image, a text, audio information, or the like, through a speaker unit (not illustrated in the present drawing). Therefore, the user may easily determine a moisture contained state in the product container P with reference to the output humidity information.

According to the container inspection device 200 as described above, the moisture contained state in the product container P may be determined through a simple manipulation, and the GMR element 410 and the moisture sensing film 430 that may be mass-produced at a low cost may be used as the humidity sensor 400 to improve productivity. In addition, the reflectance and the quality factor having a close correlation with the attenuation coefficient are set as moisture detecting reference values, thereby making it possible to improve reliability of an inspection result.

The package damage inspection device and the package damage inspection system as described above are not limited to the configurations and the methods of the exemplary embodiments described above, but all or some of the exemplary embodiments may be selectively combined with each other so that various modifications may be made.

In addition, although the spirit and scope of the present invention have been described in detail according to the exemplary embodiments, it is to be noted that the exemplary embodiments are provided in order to describe the present invention rather than limiting the present invention. Further, it may be understood by those skilled in the art to which the present invention pertains that various exemplary embodiments are possible without departing from the spirit and scope of the present invention.

1. A package damage inspection device comprising:
a sensor recognizing internal environmental change information of a sealed container;
an identification element including an identification code of the sealed container;
a recognizing unit recognizing the identification code included in the identification element to recognize identification information of the sealed container; and
a determining unit comparing the internal environmental change information recognized by the sensor and reference change information with each other to determine whether or not the sealed container is maintained in a sealed state.

2. The package damage inspection device of claim 1, wherein the determining unit compares the internal environmental change information recognized by the sensor and the reference change information with each other to determine whether or not a change in an internal environment of the sealed container exists, and determines that the sealed container is not maintained in the sealed state in the case in which it is determined that the change in the internal environment exists.

3. The package damage inspection device of claim 1, wherein the determining unit compares internal environmental change information generated in a current measuring step and internal environmental change information generated in the previous measuring step with each other to determine that the sealed container is not maintained in the sealed state.

4. The package damage inspection device of claim 1, wherein the determining unit compares internal environmental change information generated in a current measuring step and current external environment information with each other to determine whether or not the sealed container is maintained in the sealed state.

5. The package damage inspection device of claim 1, wherein the internal environmental change information is at least one of temperature information, humidity information, information on whether or not a specific material included in sealing exists, and concentration information of a material included in the sealing.

6. The package damage inspection device of claim 1, wherein the sensor is provided in the container, and periodically transmits the internal environmental change information to the determining unit through a communication unit or transmits the internal environmental change information to the determining unit through the communication unit whenever a request signal is input.

7. The package damage inspection device of claim 1, further comprising an information generating unit generating at least one of internal environmental change information for each identification code and each distributing step, decision result information on whether or not an internal environmental change exists, external environment information at the time of performing measurement, information on a measurement day and time, and information on a measurer.

8. The package damage inspection device of claim 7, wherein the information generating unit transmits the generated information and a warning message to an external device through a communication unit.

9. The package damage inspection device of claim 1, wherein the identification element is any one of a bar code, a quick response (QR) code, and a radio frequency identification (RFID) code, and
the recognizing unit is a device recognizing any one of the bar code, the QR code, and the RFID code.

10. The package damage inspection device of claim 1, further comprising an electromagnetic wave generating unit generating an electromagnetic wave,
wherein the sensor is provided in the sealed container, and changes an electromagnetic wave incident thereto depending on an internal environmental change of the sealed container to generate the changed electromagnetic wave, and
the determining unit compares the electromagnetic wave generated from the sensor and a reference electromagnetic wave corresponding to the identification code with each other to determine whether or not the sealed container is maintained in the sealed state.

11. The package damage inspection device of claim 1, wherein the reference change information has reference change information different from each other in each of a first distributing step, a second distributing step, and an N-th distributing step, and
the determining unit compares the internal environmental change information recognized by the sensor and reference change information corresponding to the identification code of the sealed container and corresponding to a current distributing step with each other to determine whether or not the sealed container is maintained in the sealed state.

12. The package damage inspection device of claim 7, further comprising a writing unit writing at least one of the internal environmental change information for each identification code and each distributing step, decision result information on whether or not the sealed container is maintained in the sealed state, the external environment information at the time of performing the measurement, the information on the measurement day and time, and the information on the measurer in the identification element,
wherein the recognizing unit recognizes the information included in the identification element.

13. A package damage inspection device comprising:
a sensor recognizing internal environmental change information of a sealed container;
an identification element for a terahertz wave including m identification units, the units including a terahertz wave transmission layer being made of a material transmitting a terahertz wave, and a waveguide diffraction grating resonating at a natural resonant frequency when the transmitted terahertz wave is radiated, in which the natural resonant frequency is any one of a first natural resonant frequency to an n-th natural resonant frequency;
a recognizing unit recognizing the identification code included in the identification element for a terahertz wave to recognize identification information of the sealed container; and
a determining unit comparing the internal environmental change information recognized by the sensor and reference change information with each other to determine whether or not the sealed container is maintained in a sealed state.

14. The package damage inspection device of claim 13, further comprising a light source irradiating the terahertz wave to the identification element for a terahertz wave,
wherein the recognizing unit detects unique resonance frequencies of the respective terahertz waves generated from the respective identification elements for a terahertz wave, and recognizes the identification code on the basis of the detected unique resonance frequencies.

15. The package damage inspection device of claim 13, wherein the determining unit compares the internal environmental change information recognized by the sensor and the reference change information with each other to determine whether or not a change in an internal environment of the sealed container exists, and determines that the sealed container is not maintained in the sealed state in the case in which it is determined that the change in the internal environment exists.

16. The package damage inspection device of claim 13, wherein the determining unit compares internal environmental change information generated in a current measuring step and internal environmental change information generated in the previous measuring step with each other to determine that the sealed container is not maintained in the sealed state.

17. The package damage inspection device of claim 13, wherein the determining unit compares internal environmental change information generated in a current measuring step and current external environment information with each other to determine whether or not the sealed container is maintained in the sealed state.

18. A package damage inspection system comprising:
a package damage inspection device including a sensor recognizing internal environmental change information of a sealed container, an identification element including an identification code of the sealed container, a recognizing unit recognizing the identification code included in the identification element to recognize identification information of the sealed container, and a determining unit comparing the internal environmental change information recognized by the sensor and reference change information with each other to determine whether or not the sealed container is maintained in a sealed state; and
a server receiving information on whether or not the sealed container is maintained in the sealed state from the package damage inspection device and storing the received information on whether or not the sealed container is maintained in the sealed state in a storing unit or transmitting the received information on whether or not the sealed container is maintained in the sealed state to a terminal of a manager.

19. The package damage inspection system of claim 18, wherein the package damage inspection device is provided in each of a first distributing step, a second distributing step, and an N-th distributing step, and
the server receives the information on whether or not the sealed container is maintained in the sealed state, internal environmental change information for each identification code and each distributing step, external environment information at the time of performing measurement, information on a measurement day and time, and information on a measurer from the package damage inspection device in each of the first distributing step, the second distributing step, and the N-th distributing step.

20. The package damage inspection system of claim 18, wherein the determining unit compares the internal environmental change information recognized by the sensor and reference change information corresponding to the identification code of the sealed container and corresponding to a current distributing step with each other to determine whether or not the sealed container is maintained in the sealed state.
